(19) United States
(12) Patent Application Publication
(10) Pub. No.: US 2016/0287642 A1
(43) Pub. Date: Oct. 6, 2016

(54) METHODS OF TREATING OR PREVENTING A LUNG DISORDER

(71) Applicant: WOMEN AND INFANTS HOSPITAL OF RHODE ISLAND, Providence, RI (US)

(72) Inventor: Monique E. DEPAEPE, Barrington, RI (US)

(73) Assignee: WOMEN AND INFANTS HOSPITAL OF RHODE ISLAND, Providence, RI (US)

(21) Appl. No.: 15/036,618

(22) PCT Filed: Nov. 14, 2014
(86) PCT No.: PCT/US14/65642

§ 371 Date: May 13, 2016

Related U.S. Application Data
(60) Provisional application No. 61/904,568, filed on Nov. 15, 2013.
(60) Provisional application No. 62/046,307, filed on Sep. 5, 2014.

(51) Int. Cl. A61K 35/51; A61K 45/06; A61K 9/00

(52) U.S. Cl. CPC A61K 35/51; A61K 9/0019; A61K 45/06

(57) ABSTRACT

Disclosed herein are methods for preventing or treating a lung disorder using, in part, mesenchymal stem cells derived from umbilical cord tissue. The methods and uses described herein relate to the administration of or use of mesenchymal stem cells, specifically those isolated and/or enriched from umbilical cord tissue, to a subject in need thereof having a lung disorder.

### CROSS-REFERENCE TO RELATED APPLICATION

This application claims benefit under 35 U.S.C. §119(e) of U.S. Provisional Application No. 61/904,568 filed Nov. 15, 2013, and U.S. Provisional Application No. 62/046,307 filed Sep. 5, 2014, the contents of each of which are incorporated herein by reference in their entirety.

### TECHNICAL FIELD

The present invention relates to novel methods for the treatment of lung disorders using umbilical cord tissue-derived mesenchymal stem cells.

### BACKGROUND

The incidence of premature delivery in the United States is currently between 11% and 12%. While programmatic efforts at reducing late preterm births have shown regional and national success, the incidence of extremely low birth weight infants has remained unchanged. Improved survival has resulted in an increased number of infants at risk for complications of prematurity. Premature infants with structurally immature lungs born between 23 and 28 weeks gestation are at risk for bronchopulmonary dysplasia (BPD), or chronic lung disease of the preterm newborn, a complex condition associated with high perinatal morbidity and mortality. In spite of increased use of exogenous surfactant and antenatal steroids, improved ventilatory strategies, and changes in neonatal intensive care, the proportion of surviving infants with BPD has remained unchanged between 1995 and 2006. An estimated 30% of very low birth weight infants (less than 1,500 g) will develop BPD and are predisposed to its long term complications, including asthma, emphysema, and poor neurodevelopmental outcome. The risk is even higher at younger gestational ages. The main pathological hallmark of BPD is an arrest of alveolar development, characterized by large, simplified distal airspaces and dysmorphic microvasculature.

### SUMMARY

The invention described herein is based, in part, on the discovery that intraperitoneal (IP) administration of mesenchymal stem cells (MSCs) derived from human umbilical cord tissue can restore normal compliance in injured lungs, such as neonatal injured lungs.

Accordingly, in one aspect, a method is provided herein for treating or preventing a lung disorder in a subject in need thereof, the method comprising administering a therapeutically effective amount of a population of isolated or enriched umbilical cord tissue-derived mesenchymal stem cells to said subject via a systemic route.

In some embodiments, the systemic route is intraperitoneal administration.

In some embodiments, the systemic route is intravenous injection.

In some embodiments, the lung disorder is a chronic lung disease.

In some embodiments, the lung disorder is emphysema.

In some embodiments, the lung disorder is a chronic lung disease of the newborn.

In some embodiments, the subject is an infant or a preterm infant.

In some embodiments, the method further comprises selecting a subject who is suffering from a lung disorder prior to administering the population of isolated or enriched umbilical cord tissue derived-mesenchymal stem cells to the subject.

In some embodiments, the population of isolated or enriched umbilical cord tissue-derived mesenchymal stem cells are expanded or cultured ex vivo prior to administration to the subject.

In some embodiments, the mesenchymal stem cells are selected based on positive expression of one or more of CD73, CD90, and CD105.

In some embodiments, the mesenchymal stem cells are selected based on negative expression of one or more of CD34, CD45, CD14, CD19, and HLA-DR.

In some embodiments, the population of isolated or enriched umbilical cord tissue-derived mesenchymal stem cells are autologous cells.

In some embodiments, the population of isolated or enriched umbilical cord tissue-derived mesenchymal stem cells are allogeneic cells obtained from one or more donors.

In some embodiments, the method further comprises administering at least one therapeutic agent.

In some embodiments, the at least one therapeutic agent enhances homing, engraftment, or survival of the population of isolated or enriched umbilical cord tissue-derived mesenchymal stem cells.

In some embodiments, the population of isolated or enriched umbilical cord tissue-derived mesenchymal stem cells is isolated, enriched, or expanded from human umbilical cord perivascular cells.

In some embodiments, the population of isolated or enriched umbilical cord tissue-derived mesenchymal stem cells is isolated, enriched, or expanded from Wharton's jelly.

### BRIEF DESCRIPTION OF THE DRAWINGS

FIGS. 1A-1G show analysis of alveolarization.

FIG. 1A is a representative micrograph of normoxia-exposed control animal at 9 weeks of age showing a complex alveolar network. H&E staining, original magnification: ×200.

FIG. 1B is a representative micrograph of hyperoxia-exposed control animal, exposed to 90% O₂ from birth until P7, examined at 9 weeks of age. The airspaces are large and simplified, replicating the emphysema-like morphology of ‘new BPD’. H&E staining, original magnification: ×200.

FIG. 1C is a representative micrograph of hyperoxia-exposed animal treated with 1×10⁶ MSC via intranasal route. H&E staining, original magnification: ×200.

FIG. 1D is a representative micrograph of hyperoxia-exposed animal treated with 1×10⁶ MSC via intraperitoneal route. H&E staining, original magnification: ×200.

FIGS. 1E-1G show morphometric analysis of lungs 8 weeks post-transplantation of 1×10⁶ MSCs to hyperoxia-exposed newborn mice via intranasal or intraperitoneal delivery. Controls were PBS-treated normoxic and hyperoxic animals. Values represent mean±SD of at least 6 animals per group. AA(ae/lu): areal density of air-exchanging lung parenchyma. *: P<0.05; **: P<0.01; ***: P<0.0001.

FIG. 2 shows analysis of lung mechanics. Pulmonary function tests 8 weeks post-transplantation of 1×10⁶ MSCs to hyperoxia-exposed newborn mice via intranasal (IN) or intraperitoneal (IP) delivery. Controls were PBS-treated normoxic and hyperoxic animals. Presented are selected data obtained by snapshot (Crs) and quickprime-3 (H) perturbations and maximal PV loops (A and hysteresis (area between inflating and deflating part of the loop) by FlexiVent technique in tracheotomized mice. Boxplot analyses represent group median, upper and lower quartiles (box), maximum and minimum values excluding outliers (whiskers), and outliers (more than 3/2 times upper quartile) (bullets). At least 6 animals were studied per group. See also Tables 3 and 4 for additional data. *: P<0.05; **: P<0.01; ****: P<0.0001 versus normoxic controls. °: P<0.05; °°: P<0.01 versus hyperoxic controls.

FIG. 3 shows analysis of lung mechanics: Pressure-volume (PV) loops—Pressure-regulated (PVr-P) PV-loops were generated using the PVr-P perturbation from the FlexiVent. Figures represent mean±SD of at least 6 animals per pressure point for normoxic controls (dashed lines), hyperoxic controls (dash-dot lines), or MSC-treated hyperoxic controls (solid lines).

FIGS. 4A-4F show analysis of MSC distribution 48 hours after intranasal or intraperitoneal administration. In FIGS. 4A, 4B, 4D, and 4F, avidin-biotin peroxidase staining, hematoxylin counterstain. In FIGS. 4C and 4E, H&E staining.

FIG. 4A shows representative anti-human vimentin staining of lungs of animal treated with 1×10⁶ MSCs IN. Numerous human vimentin-positive single or aggregated MSCs are seen distributed in peribronchial and more distal lung parenchyma (arrows). Murine mesenchymal cells, such as fibroblasts, endothelial cells, and peribronchial/perivascular smooth muscle cells, show no cross-reactivity with the anti-human vimentin antibody, supporting its specificity for human cells. (Original magnification: ×200).

FIG. 4B shows anti-human vimentin staining of intestinal tract of animal treated with 1×10⁶ MSCs IN. A single MSC is seen in the lumen (arrow), consistent with occasional spillage of very small numbers of intranasally delivered cells into gastrointestinal tract. (Original magnification: ×200).

FIGS. 4C-4D are micrographs showing anti-human vimentin staining of pancreas and peripancreatic soft tissue of animal treated with 1×10⁶ MSCs IP. Isolated and clustered MSCs are embedded in the peripancreatic soft tissue, associated with mild mesothelial and stromal reactive changes. (Original magnification: ×200)

FIGS. 4E-4F are micrographs showing anti-human vimentin staining of perisplenic soft tissue, possibly omentum, of animal treated with 1×10⁶ MSCs IP. A large-sized nodular aggregate of MSCs is seen protruding from the soft tissue. Several scattered smaller MSC aggregates are noted. (Original magnification: ×100)

FIGS. 5A-5C show analysis of engraftment and proliferation of human MSC-derived cells. Confocal fluorescence microscopy of lungs subjected to combined anti-Ki67 immunofluorescence visualized as red and alu-FISH analysis visualized as green and DAPI counterstain visualized as blue.

FIG. 5A is a representative micrograph of lungs of animal treated with 1×10⁶ MSCs IN showing a doublet of alu-FISH-positive cells (green) along the alveolar wall. Several proliferating FISH-negative murine cells are noted as visualized as red.

FIG. 5B is a representative micrograph of lungs of animal treated with 1×10⁶ MSCs IN showing a proliferating alu-FISH positive cell visualized as yellow-orange along the alveolar wall. Cytoplasmic granular green autofluorescence noted in several large-sized cells is consistent with presence of hemosiderin pigment in murine alveolar macrophages.

FIG. 5C is a representative micrograph of lungs of animal treated with 1×10⁶ MSCs IP showing a doublet of alu-FISH-positive cells visualized as green along the alveolar wall.

FIGS. 6A-6F show analysis of bronchoalveolar lavage fluid.

FIGS. 6A-6D show representative morphology of alveolar macrophages. FIG. 6A: Normoxia, PBS control; FIG. 6B: hyperoxia, PBS control; FIG. 6C: hyperoxia, MSC high IN; FIG. 6D: hyperoxia, MSC high, IP. Cytoplasmic granules are more frequent and conspicuous in alveolar macrophages of hyperoxia-exposed animals than in normoxic controls. (Giemsa stain, magnification ×1,000 (oil)).

FIG. 6E is a micrograph showing Perls iron staining of lavage fluid of hyperoxia-exposed control animal showing two alveolar macrophages with abundant cytoplasmic hemosiderin granules.

FIG. 6F is a plot showing fraction of granule-containing alveolar macrophages, expressed as a percentage. Values represent mean±SD of at least 6 animals per group. *: P<0.01; **: P<0.0001 versus normoxic PBS-treated controls. °: P<0.001 versus hyperoxic PBS-treated controls.

### DETAILED DESCRIPTION

The invention described herein generally relates to new and enhanced methods for improving or restoring the function of injured lungs using mesenchymal stem cells, particularly those derived from umbilical cord tissue. Further, the inventor has discovered that administration route and dosage of stem cells are important factors in achieving desired therapeutic effects. Specifically, the inventor has found that systemic administration (e.g., intraperitoneal) can restore normal compliance in injured lungs, such as neonatally injured lungs, while intranasal delivery has no obvious pulmonary effects.

Accordingly, provided herein, in part, are methods for the treatment or prevention of a respiratory disease or disorder in a subject in need thereof. The treatment methods described herein can be used in a subject of any age, such as an adult, a young subject, an infant, and a newborn. The methods described herein involve, in part, administration of therapeutically effective amounts of mesenchymal stem cells, particularly those derived from umbilical cord tissue, to subjects having respiratory diseases or disorders.

Stem cells are cells that retain the ability to renew themselves through mitotic cell division and can differentiate into a diverse range of specialized cell types. The two broad types of mammalian stem cells are: embryonic stem (ES) cells that are found in blastocysts, and adult stem cells that are found in adult tissues. In a developing embryo, stem cells can differentiate into all of the specialized embryonic tissues. In adult organisms, stem cells and progenitor cells act as a repair system for the body, replenishing specialized cells, but also maintain the normal turnover of regenerative organs, such as blood, skin or intestinal tissues. Pluripotent stem cells can differentiate into cells derived from any of the three germ layers.

### Mesenchymal Stem Cells

As used herein, the terms “mesenchymal stem cell”, “mesenchymal stromal cell”, or abbreviated “MSC” refer to a generalized cell that has multipotency (descendants can specialize into different cell types), for example, an undifferentiated MSC that is capable of differentiating into more than one specific type of mesoderm-derived cells and regenerating into various tissues in vivo. Such cells also have unlimited proliferating and self-renewal capability and can differentiate into osteogenic, myogenic, adipogenic or chondrogenic, neurogenic, hepatogenic, nephrogenic, urogenic, isletogenic, pancreatogenic, gastroenterogenic, epitheliogenic, thyroidogenic, myocardiogenic, pneumogenic, retinogenic, gametogenic, endotheliogenic, or hematopoietic lineages.

The mesenchymal stem cells can be selected based on positive or negative expression of one or more markers. In some embodiments, the mesenchymal stem cells are selected based on positive expression of one or more of CD73, CD90, and CD105. In some embodiments, the mesenchymal stem cells express HLA class I and one or more of CD49c, CD49d, CD49e, and CD49f. In some embodiments, the mesenchymal stem cells are selected based on negative expression of one or more of CD34, CD45, CD14, CD19, and HLA-DR. In some embodiments, the mesenchymal stem cells used in the methods described herein are selected for, enriched for, or isolated using one or more of these additional cell surface markers.

In some embodiments, where the mesenchymal stem cells are obtained from umbilical cord tissue, for example, the mesenchymal stem cells are positive for CD105 (SH2), CD73 (SH3), CD90 (Thy-1), and CD44, but negative for CD45, CD34, CD235a (glycophorin A), CD106 (VCAM1), CD123 (IL3), SSEA-4, HLA-DR, DP, DQ (MHCII), HLA-G, and Oct4.

The mesenchymal stem cells used for the various aspects described herein can be derived or isolated from any one or more of the following sources: umbilical cord tissue, umbilical cord blood, placental tissue, bone marrow, adipose tissue, peripheral blood mononuclear cells, differentiated embryonic stem cells, and differentiated progenitor cells.

In some embodiments, the cells from the biological sources described herein can be expanded ex vivo using any method acceptable to those skilled in the art prior to use in the methods described herein. Further, the cells can be sorted, fractionated, treated to remove unwanted or malignant cells, or otherwise manipulated to treat the patient using any procedure acceptable to those skilled in the art of preparing cells for administration.

As used herein, the term “population of mesenchymal cells” encompasses a heterogeneous or homogeneous population of mesenchymal stem cells and/or mesenchymal progenitor cells. In addition, differentiated mesenchymal cells can be present in a population of mesenchymal cells. A population of mesenchymal cells comprising at least two different cell types is referred to herein as a “heterogeneous population”. It is also contemplated herein that mesenchymal stem cells or mesenchymal progenitor cells are isolated and expanded ex vivo prior to administration. A population of mesenchymal cells comprising only one cell type (e.g., mesenchymal stem cells) is referred to herein as a “homogeneous population of cells”.

Attractive properties of MSCs in this context include, but are not limited to, their capacity to specifically home to injured tissue and to exert immunomodulatory activities with secretion of anti-inflammatory factors (e.g. interferon-γ, interleukin-10, vascular endothelial growth factor, hepatocyte growth factor); angiogenic factors; and anti-apoptotic factors. Exogenously administered MSCs may exert their effects by both cell contact-dependent and paracrine mechanisms involving secretion of specific mediators and transfer of cellular materials such as proteins, nucleic acids, and cellular organelles (including mitochondria) to host cells via microvesicles (Fung M E, Thebaud B: Stem cell-based therapy for neonatal lung disease: it is in the juice, Pediatr Res 2014, 75:2-7).

### Isolation of MSCs

Mesenchymal stem cells for use in the methods and uses described herein can be enriched for or isolated from a biological sample, preferably umbilical cord tissue, using any method known to one of skill in the art.

The term “biological sample” as used herein refers to a cell or population of cells or a quantity of tissue or fluid from a subject comprising one or more mesenchymal stem cells. Most often, the biological sample has been removed from a subject, but the term “biological sample” can also refer to cells or tissue analyzed in vivo, i.e., without removal from the subject. Biological samples include, but are not limited to, umbilical cord blood, umbilical cord tissue, whole blood, bone marrow, tissue sample or biopsies, scrapes (e.g. buccal scrapes), plasma, serum, urine, saliva, cell culture, or cerebrospinal fluid.

In some embodiments of the aspects described herein, a biological sample comprising mesenchymal stem cells refers to a sample isolated from a subject, such as umbilical cord tissue, umbilical cord blood, peripheral blood, thymus, or bone marrow, which is then further processed, for example, by cell sorting (e.g., magnetic sorting or FACS), to obtain a population of mesenchymal stem cells. In other embodiments of the aspects described herein, a biological sample comprising mesenchymal stem cells refers to an in vitro or ex vivo culture of expanded mesenchymal stem cells.

In some embodiments, a biological sample comprising MSCs can undergo an enzymatic digestion step. A collagenase and/or another protease, such as a hyaluronidase and/or a dispase, can be used to digest the biological sample comprising MSCs. For example, a cord tissue can undergo overnight digestion in collagenase (e.g., collagenase NB 6, GMP grade, 0.75 mg/ml, Serva, Heidelberg, Del.) with antibiotics in a CaCl₂-buffered digestion solution (37° C.). In another example, tissue digestion may be facilitated by acids. Such digestion of umbilical cord tissue, for example, results in a heterogenous population of cells comprising, for example, epithelial cells, endothelial cells, arterial cells, periocytes, and mesenchymal stem cells.

In some embodiments, a biological sample comprising MSCs, such as an umbilical cord tissue, can undergo enzymatic digestion and processing as described in “Human Umbilical Cord Perivascular (HUCPV) Cells: A Source of Mesenchymal Progenitors,” Stem Cells 2005; 23:220-229, the contents of which are herein incorporated by reference in their entireties. Briefly, pieces of umbilical cord (UC) tissue, 4-5 cm long, are dissected by first parting the epithelium of the UC section along its length to expose the underlying Wharton's Jelly (WJ). Each vessel, with its surrounding WJ matrix, is pulled away, and the ends of each dissected vessel tied together with a suture creating “loops” that are placed into a tube containing a solution of 1 mg/ml collagenase with phosphate buffered saline (PBS). After 18-24 hours, the loops are removed from the suspension, which is then diluted with PBS to reduce the viscosity of the suspension and centrifuged. Following the removal of the supernatant, the cells are resuspended in PBS. The suspended cells are depleted of hematopoietic cells, for example using magnetic beads. Cells are plated in tissue culture polystyrene dishes supplemented medium (SM) (75% α-MEM, 15% fetal bovine serum [FBS]), and 10% antibiotics, which is changed every 2 days.

In some embodiments, the MSCs or heterogenous population of cells obtained after the enzymatic digestion step can be used directly in administration. In some embodiments, the MSCs obtained after the enzymatic digestion step can be further expanded prior to administration.

In some embodiments of the aspects described herein, the mesenchymal stem cells are isolated prior to their administration to a subject in need thereof. Such isolation can result in a substantially pure or enriched cell population for administration to the subject.

The terms “isolate” and “methods of isolation,” as used herein, refer to any process whereby a cell or population of cells, such as a population of mesenchymal stem cells, is removed from a subject or sample in which it was originally found, or a descendant of such a cell or cells. The term “isolated population,” as used herein, refers to a population of cells that has been removed and separated from a biological sample, or a mixed or heterogeneous population of cells found in such a sample. Such a mixed population includes, for example, a population of mesenchymal stem cells obtained from umbilical cord tissue. In some embodiments, an isolated population is a substantially pure population of cells as compared to the heterogeneous population from which the cells were isolated or enriched from. In some embodiments of this aspect and all aspects described herein, the isolated population is an isolated population of mesenchymal stem cells. In other embodiments of this aspect and all aspects described herein, the isolated population comprises a substantially pure population of mesenchymal stem cells as compared to a heterogeneous population of cells comprising various other cells types from which the mesenchymal stem cells were derived. In some embodiments, an isolated cell or cell population, such as a population of mesenchymal stem cells, is further cultured in vitro or ex vivo, e.g., in the presence of growth factors or cytokines, to further expand the number of cells in the isolated cell population or substantially pure cell population. Such culture can be performed using any method known to one of skill in the art, for example, as described in the Examples section. In some embodiments, the isolated or substantially pure mesenchymal stem cells populations obtained by the methods disclosed herein are later administered to a second subject, or re-introduced into the subject from which the cell population was originally isolated (e.g., allogenic transplantation vs. autologous administration).

The term “substantially pure,” with respect to a particular cell population, refers to a population of cells that is at least about 75%, at least about 80%, at least about 85%, at least about 90%, at least about 95%, at least about 98%, or at least about 99% pure, with respect to the cells making up a total cell population. In other words, the terms “substantially pure” or “essentially purified,” with regard to a population of mesenchymal stem cells isolated for use in the methods disclosed herein, refers to a population of mesenchymal stem cells that contain fewer than about 25%, fewer than about 20%, fewer than about 15%, fewer than about 10%, fewer than about 9%, fewer than about 8%, fewer than about 7%, fewer than about 6%, fewer than about 5%, fewer than about 4%, fewer than about 3%, fewer than about 2%, fewer than about 1%, of cells that are not mesenchymal stem cells, as defined by the terms herein. Some embodiments of these aspects further encompass methods to expand a population of substantially pure or enriched mesenchymal stem cells, wherein the expanded population of mesenchymal stem cells is also a substantially pure or enriched population of mesenchymal stem cells.

The terms “enriching” or “enriched” are used interchangeably herein and mean that the yield (fraction) of cells of one type, such as mesenchymal stem cells for use in the methods described herein, is increased by at least 15%, by at least 20%, by at least 25%, by at least 30%, by at least 35%, by at least 40%, by at least 45%, by at least 50%, by at least 55%, by at least 60%, by at least 65%, by at least 70%, or by at least 75%, over the fraction of cells of that type in the starting biological sample, culture, or preparation. A population of mesenchymal stem cells obtained for use in the methods described herein is most preferably at least 60% enriched for mesenchymal stem cells.

In some embodiments of the aspects described herein, markers specific for mesenchymal stem cells are used to isolate or enrich for these cells. A “marker,” as used herein, describes the characteristics and/or phenotype of a cell. Markers can be used for selection of cells comprising characteristics of interest. Markers will vary with specific cells. Markers are characteristics, whether morphological, functional or biochemical (enzymatic), particular to a cell type, or molecules expressed by the cell type. Preferably, such markers are proteins, and more preferably, possess an epitope for antibodies or other binding molecules available in the art. However, a marker may consist of any molecule found in a cell including, but not limited to, proteins (peptides and polypeptides), lipids, polysaccharides, nucleic acids and steroids. Examples of morphological characteristics or traits include, but are not limited to, shape, size, appearance (e.g., smooth, translucent), and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate along particular lineages. Markers may be detected by any method available to one of skill in the art.

Accordingly, as used herein, a “cell-surface marker” refers to any molecule that is expressed on the surface of a cell. Cell-surface expression usually requires that a molecule possesses a transmembrane domain. Some molecules that are normally not found on the cell-surface can be engineered by recombinant techniques to be expressed on the surface of a cell. Many naturally occurring cell-surface markers are termed “CD” or “cluster of differentiation” molecules. Cell-surface markers often provide antigenic determinants to which antibodies can bind to. The useful mesenchymal stem cells according to the present invention preferably express one or more of CD73, CD90, and CD105 and/or CD44.

In some embodiments of the aspects described herein, a variety of methods to isolate a substantially pure or enriched population of mesenchymal stem cells are available to a skilled artisan, including immunoselection techniques, such as high-throughput cell sorting using flow cytometric methods, affinity methods with antibodies labeled to magnetic beads, biodegradable beads, non-biodegradable beads, and antibodies panned to surfaces including dishes, and any combination of such methods.

In some embodiments of these aspects and all aspects described herein, isolation of and enrichment for populations of mesenchymal stem cells can be performed using bead based sorting mechanisms, such as magnetic beads. In such methods, the biological sample, such as umbilical cord tissue, is contacted with magnetic beads coated with antibodies against one or more specific cell-surface antigens, such as CD73, CD90, and CD105. This causes the cells in the sample expressing this antigen to attach to the magnetic beads. Afterwards the contacted cell solution is transferred to a strong magnetic field, such as a column or rack having a magnet. The cells attached to the beads (expressing the cell-surface marker) stay on the column or sample tube, while other cells (not expressing the cell-surface marker) flow through or remain in solution. Using this method, cells can be separated positively or negatively, or using a combination therein, with respect to the particular cell-surface markers.

In some embodiments of the aspects described herein, magnetic activated cell sorting (MACS) strategies are used for isolation and preselection of mesenchymal stem cells. In some such embodiments, the isolated mesenchymal stem cells are still coupled with the microbead-bound antibodies when administered to a subject in need. In some embodiments, mesenchymal stem cells are isolated in the presence of human plasma or human serum albumin (HSA), such as 2% HSA.

In some preferred embodiments of the aspects described herein, MSCs are isolated or enriched using positive selection for one or more of the cell-surface markers CD73, CD90, and CD105 and/or CD44.

In other embodiments, one or more additional cell-surface markers are used for isolating and/or enriching for MSCs, using positive or negative selection methods, or a combination therein. In some embodiments, the mesenchymal stem cells are selected based on negative expression of one or more of CD34, CD45, CD14, CD19, and HLA-DR.

As defined herein, “positive selection” refers to techniques that result in the isolation or enrichment of cells expressing specific cell-surface markers, while “negative selection” refers to techniques that result in the isolation or enrichment of cells not expressing specific cell-surface markers. In some embodiments, beads can be coated with antibodies by a skilled artisan using standard techniques known in the art, such as commercial bead conjugation kits. In some embodiments, a negative selection step is performed to remove cells expressing one or more lineage markers, followed by fluorescence activated cell sorting to positively select mesenchymal stem cells expressing one or more specific cell-surface markers. For example, in a negative selection protocol, a biological sample, such as a cell sample, is first contacted with labeled antibodies specific for cell-surface markers of interest, such as CD34, CD45, CD14, CD19, and HLA-DR, and the sample is then contacted with beads that are specific for the labels of the antibodies, and the cells expressing any of the markers CD34, CD45, CD14, CD19, and HLA-DR are removed using immunomagnetic lineage depletion.

A number of other surface markers can be used in the isolation and/or enrichment of MSCs, such as HLA class I, CD49c, CD49d, CD49e, CD49f, CD44, CD146, CD271, CD11b, CD31, and CD144. A review on the surface markers of human MSCs can be found, for example, in Lv et al., Stem Cells 2014, 32, 1408-1419, the contents of which are incorporated by reference.

Other embodiments of the aspects described herein use flow cytometric methods, alone or in combination with magnetic bead based methods, to isolate or enrich for hematopoetic stem cells. As defined herein, “flow cytometry” refers to a technique for counting and examining microscopic particles, such as cells and chromosomes, by suspending them in a stream of fluid and passing them through an electronic detection apparatus. Flow cytometry allows simultaneous multiparametric analysis of the physical and/or chemical parameters of up to thousands of particles per second, such as fluorescent parameters. Modern flow cytometric instruments usually have multiple lasers and fluorescence detectors. Increasing the number of lasers and detectors allows for labeling by multiple antibodies, and can more precisely identify a target population by their phenotypic markers. Certain flow cytometric instruments can take digital images of individual cells, allowing for the analysis of fluorescent signal location within or on the surface of cells.

A common variation of flow cytometric techniques is to physically sort particles based on their properties, so as to purify populations of interest, using “fluorescence-activated cell sorting” As defined herein, “fluorescence-activated cell sorting” or “flow cytometric based sorting” methods refer to flow cytometric methods for sorting a heterogeneous mixture of cells from a single biological sample into one or more containers, one cell at a time, based upon the specific light scattering and fluorescent characteristics of each cell and provides fast, objective and quantitative recording of fluorescent signals from individual cells as well as physical separation of cells of particular interest. Accordingly, in those embodiments when the agents specific for cell-surface markers are antibodies labeled with tags that can be detected by a flow cytometer, fluorescence-activated cell sorting (FACS) can be used in and with the methods described herein to isolate and enrich for populations of mesenchymal stem cells.

Some methods of isolating MSCs from various sources are disclosed, for example, in US20120142102, U.S. Pat. No. 7,592,174, WO2014053420, US20130156819, and US20120294837, the contents of each of which are incorporated by reference in their entirety.

### Expansion of MSCs

In some embodiments of the aspects, the substantially pure or enriched for population of isolated mesenchymal stem cells are further expanded or increased in numbers prior to their use in the methods of treatment and uses described herein.

In some embodiments, mesenchymal stem cells isolated or enriched for using the methods and techniques described herein are expanded in culture, i.e., the cell numbers are increased, using methods known to one of skill in the art, prior to administration to a subject in need. In some embodiments, such expansion methods can comprise, for example, culturing the mesenchymal stem cells in serum-free medium supplemented with factors and/or under conditions that cause expansion of mesenchymal stem cells, or combinations thereof.

Some methods of expanding MSCs are disclosed, for example, in US20070298497, US20140023623, US20110129918, US20100047211, WO2013121426, and WO2010110768, the contents of each of which are incorporated by reference in their entirety.

In some embodiments, the mesenchymal stem cells are expanded using the methods described in “Human Umbilical Cord Perivascular (HUCPV) Cells: A Source of Mesenchymal Progenitors,” Stem Cells 2005; 23:220-229. For example, cells can be plated in tissue culture polystyrene dishes in supplemented medium (SM) (75% α-MEM, 15% fetal bovine serum [FBS]), and 10% antibiotics, which is changed every 2 days. At day 7, adherent cells, judged 80%-90% confluent by phase contrast microscopy, are passaged using 0.1% trypsin solution and plated in tissue culture polystyrene flasks at, for example, 4×10³ cells/cm² in SM.

In some embodiments, the mesenchymal stem cells are expanded until a therapeutically effective number of cells is achieved, for example, a population of up to 1 million, up to 10 million, up to 50 million, up to 100 million, or up to 200 million cells.

The terms “increased,” “increase,” “enhance,” or “expand” are all used herein to generally mean an increase in the number of mesenchymal stem cells by a statically significant amount; for the avoidance of any doubt, the terms “increased,” “increase,” “expand,” “expanded,” or “enhance” mean an increase, as compared to a reference level, of at least about 10%, of at least about 15%, of at least about 20%, of at least about 25%, of at least about 30%, of at least about 35%, of at least about 40%, of at least about 45%, of at least about 50%, of at least about 55%, of at least about 60%, of at least about 65%, of at least about 70%, of at least about 75%, of at least about 80%, of at least about 85%, of at least about 90%, of at least about 95%, or up to and including a 100%, or at least about a 2-fold, or at least about a 3-fold, or at least about a 4-fold, or at least about a 5-fold, at least about a 6-fold, or at least about a 7-fold, or at least about a 8-fold, at least about a 9-fold, at least about a 10-fold increase, at least about a 25-fold increase, at least about a 50-fold increase, at least about a 100-fold increase, or any increase of 100-fold or greater, as compared to a control or reference level. A control sample or control level is used herein to describe a population of cells obtained from the same biological source that has, for example, not been expanded using the methods described herein.

### Umbilical Cord Tissue

In preferred embodiments of the aspects described herein, human umbilical cord (UC) tissue is a source of MSCs for administration to a subject in need.

The human umbilical cord is embryologically derived at day 26 of gestation, and it grows to form a 30- to 50-cm-long helical organ at birth. During the 40 weeks of gestation, a mesenchymal precursor cell population develops within the UC that gives rise to the Wharton's jelly (WJ) connective tissue, and are located closest to the vasculature. These cells are a sub-population of the cells termed “umbilical cord perivascular (HUCPV) cells,” and thus can be expanded from HUCPV cells.

Various cord tissues can be used in the present invention, such as the vasculature including vessel walls and endothelium, umbilical cord perivascular (HUCPV) cells, the Wharton's jelly, the amniotic epithelium and the like. The cord from which such tissues are obtained can be cord from any mammal, and is preferably obtained from human umbilical cord. In one embodiment, the umbilical cord tissue comprises umbilical cord perivascular (HUCPV) cells. In one embodiment, the umbilical cord tissue is Wharton's jelly. In one embodiment, the tissue is Wharton's jelly associated with the perivascular region of umbilical cord vasculature, desirably human umbilical cord vasculature. In another embodiment, the umbilical cord tissue is vascular tissue. In yet another embodiment, the tissue is vascular tissue having Wharton's jelly associated with the perivascular region bound thereto. In a another embodiment, the umbilical cord tissue is the vasculature (i.e., vessels) and associated Wharton's jelly that remains associated therewith when the vasculature is removed from within the resected cord. Such cord tissue includes the entire length of the intact vasculature, individual vessels, longitudinally sectioned forms thereof from which blood has been optionally removed, and transverse sections of such tissues.

The cord tissue desirably is obtained fresh, as post-partum tissue, and following optional dissection to provide tissue of the nature just described above, is then prepared for freezing. Desirably, the cord tissue is processed within about 24 hours from harvest, and the tissues thus extracted are frozen, and desirably enter cryogenic storage, within at least about 72 hours from harvest, and more desirably within 48 hours and particularly 24 hours from harvest. The fresh tissue can be cooled during this period, and is desirably washed and optionally disinfected, in accordance with standard practice, but should not be frozen during this period except as noted herein, so that cell viability is not adversely affected.

In some embodiments, the umbilical cord tissue is obtained post-partum, and subjected to freezing whereby the frozen umbilical cord tissue is then stored as future source of viable cells. To obtain viable cells from the frozen tissue, the tissue is allowed to thaw and is then extracted to provide cells that, when cultured, exhibit viability.

### Administration and Uses of MSCs in Regenerative Medicine

In some embodiments of the aspects described herein, the mesenchymal stem cell population being administered according to the methods described herein, comprises allogeneic mesenchymal stem cells obtained from one or more donors. As used herein, “allogeneic” refers to mesenchymal stem cell or biological samples comprising mesenchymal stem cell obtained from one or more different donors of the same species, where the genes at one or more loci are not identical. For example, a mesenchymal stem cell population being administered to a subject can be obtained from umbilical cord tissue obtained from one or more unrelated donor subjects, or from one or more non-identical siblings or relatives. In some embodiments, syngeneic mesenchymal stem cell populations can be used, such as those obtained from genetically identical animals, or from identical twins. In other embodiments of this aspect, the mesenchymal stem cells are autologous mesenchymal stem cells. As used herein, “autologous” refers to mesenchymal stem cells or biological samples comprising mesenchymal stem cells obtained or isolated from a subject and being administered to the same subject, i.e., the donor and recipient are the same.

In some embodiments of the aspects described herein, the MSCs can be stored in a stem cell bank. The stem cell bank can provide a large pool of available stem cells, that can be utilized in a variety of therapeutic, as well as research, applications. The stored stem cells can serve, for example, as a source of cells for use in the future when health reasons require stem cells technologies to treat certain cell populations of an individual's body. The stored stem cells can also serve as a source of cells for autologous use, for example, for curing future diseases of the donor. The stored stem cells can also serve as a source of cells for curing future diseases of a relative of the donor. The stored stem cells can also serve as a source of cells for clinical use by other individuals upon authorization from the donor. In some embodiments of the aspects described herein, the MSCs can be cryopreserved for later administration.

The methods described herein can be used to treat, ameliorate, prevent or slow the progression of a number of respiratory diseases or their symptoms, such as those resulting in pathological damage to lung or airway architecture and/or alveolar damage. The terms “respiratory disorder,” “respiratory disease,” “pulmonary disease,” and “pulmonary disorder,” are used interchangeably herein and refer to any condition and/or disorder relating to respiration and/or the respiratory system, including the lungs, pleural cavity, bronchial tubes, trachea, upper respiratory tract, airways, or other components or structures of the respiratory system.

Such respiratory diseases include, but are not limited to, bronchopulmonary dysplasia (BPD), chronic obstructive pulmonary disease (COPD) condition, cystic fibrosis, bronchiectasis, cor pulmonale, pneumonia, lung abcess, acute bronchitis, chronic bronchitis, emphysema, pneumonitis, e.g., hypersensitivity pneumonitis or pneumonitis associated with radiation exposure, alveolar lung diseases and interstitial lung diseases, environmental lung disease (e.g., associated with asbestos, fumes or gas exposure), aspiration pneumonia, pulmonary hemorrhage syndromes, amyloidosis, connective tissue diseases, systemic sclerosis, ankylosing spondylitis, pulmonary actinomycosis, pulmonary alveolar proteinosis, pulmonary anthrax, pulmonary edema, pulmonary embolus, pulmonary inflammation, pulmonary histiocytosis X, pulmonary hypertension, surfactant deficiencies, pulmonary hypoplasia, pulmonary neoplasia, pulmonary nocardiosis, pulmonary tuberculosis, pulmonary veno-occlusive disease, rheumatoid lung disease, sarcoidosis, post-pneumonectomy, Wegener's granulomatosis, allergic granulomatosis, granulomatous vasculitides, eosinophilia, asthma and airway hyperreactivity (AHR), e.g., mild intermittent asthma, mild persistent asthma, moderate persistent asthma, severe persistent asthma, acute asthma, chronic asthma, atopic asthma, allergic asthma or idiosyncratic asthma, cystic fibrosis and associated conditions, e.g., allergic bronchopulmonary aspergillosis, chronic sinusitis, pancreatic insufficiency, lung or vascular inflammation, bacterial or viral infection, e.g., Haemophilus influenzae, S. aureus, Pseudomonas aeruginosa or respiratory syncytial virus (RSV) infection or an acute or chronic adult or pediatric respiratory distress syndrome (RDS) such as grade I, II, III or IV RDS or an RDS associated with, e.g., sepsis, pneumonia, reperfusion, atelectasis or chest trauma. In some embodiments, the respiratory disorder being treated is emphysema.

Chronic obstructive pulmonary diseases (COPDs) include those conditions where airflow obstruction is located at upper airways, intermediate-sized airways, bronchioles or parenchyma, which can be manifested as, or associated with, tracheal stenosis, tracheal right ventricular hypertrophy pulmonary hypertension, polychondritis, bronchiectasis, bronchiolitis, e.g., idiopathic bronchiolitis, ciliary dyskinesia, asthma, emphysema, connective tissue disease, bronchiolitis of chronic bronchitis or lung transplantation.

The methods described herein can also be used to treat or ameliorate acute or chronic asthma or their symptoms or complications, including airway epithelium injury, airway smooth muscle spasm or airway hyperresponsiveness, airway mucosa edema, increased mucus secretion, excessive, T cell activation, or desquamation, atelectasis, cor pulmonale, pneumothorax, subcutaneous emphysema, dyspnea, coughing, wheezing, shortness of breath, tachypnea, fatigue, decreased forced expiratory volume in the 1st second (FEV₁), arterial hypoxemia, respiratory acidosis, inflammation including unwanted elevated levels of mediators such as IL-4, IL-5, IgE, histamine, substance P, neurokinin A, calcitonin gene-related peptide or arachidonic acid metabolites such as thromboxane or leukotrienes (LTD₄ or LTC₄), and cellular airway wall infiltration, e.g., by eosinophils, lymphocytes, macrophages or granulocytes.

Any of these and other respiratory or pulmonary conditions or symptoms are described elsewhere, e.g., The Merck Manual, 19th edition, edited by Robert S. Porter, 2011, Merck, ISBN-10: 0911910190, or in other references cited herein. In some of these conditions, where inflammation plays a role in the pathology of the condition, the methods described herein can ameliorate or slow the progression of the condition by reducing damage from inflammation. In other cases, the methods described herein act to limit pathogen replication or pathogen-associated lung tissue damage.

When provided prophylactically, isolated or enriched mesenchymal stem cells can be administered to a subject in advance of any symptom of a respiratory disorder, e.g., asthma attack or to a premature infant. Accordingly, the prophylactic administration of an isolated or enriched for mesenchymal stem cell population serves to prevent a respiratory disorder, as disclosed herein.

When provided therapeutically, isolated or enriched mesenchymal stem cells are provided at (or after) the onset of a symptom or indication of a respiratory disorder, e.g., upon the onset of COPD.

In some embodiments of the invention, the subject is first diagnosed as having a disease or disorder affecting the lung tissue prior to administering the cells according to the methods described herein. In some embodiments, the subject is first diagnosed as being at risk of developing lung disease or disorder prior to administering the cells. For example, a premature infant may be at a significant risk of developing a lung disease or disorder.

For use in the various aspects described herein, an effective amount of mesenchymal stem cells, or an enriched fraction thereof, comprises at least 10² mesenchymal stem cells, at least 5×10² mesenchymal stem cells, at least 10³ mesenchymal stem cells, at least 5×10³ mesenchymal stem cells, at least 10⁴ mesenchymal stem cells, at least 5×10⁴ mesenchymal stem cells, at least 10⁵ mesenchymal stem cells, at least 2×10⁵ mesenchymal stem cells, at least 3×10⁵ mesenchymal stem cells, at least 4×10⁵ mesenchymal stem cells, at least 5×10⁵ mesenchymal stem cells, at least 6×10⁵ mesenchymal stem cells, at least 7×10⁵ mesenchymal stem cells, at least 8×10⁵ mesenchymal stem cells, at least 9×10⁵ mesenchymal stem cells, at least 1×10⁶ mesenchymal stem cells, at least 2×10⁶ mesenchymal stem cells, at least 3×10⁶ mesenchymal stem cells, at least 4×10⁶ mesenchymal stem cells, at least 5×10⁶ mesenchymal stem cells, at least 6×10⁶ mesenchymal stem cells, at least 7×10⁶ mesenchymal stem cells, at least 8×10⁶ mesenchymal stem cells, at least 9×10⁶ mesenchymal stem cells, at least 1×10⁷ mesenchymal stem cells, at least 2×10⁷ mesenchymal stem cells, at least 3×10⁷ mesenchymal stem cells, at least 4×10⁷ mesenchymal stem cells, at least 5×10⁷ mesenchymal stem cells, at least 6×10⁷ mesenchymal stem cells, at least 7×10⁷ mesenchymal stem cells, at least 8×10⁷ mesenchymal stem cells, at least 9×10⁷ mesenchymal stem cells, or multiples thereof. The mesenchymal stem cells can be isolated or enriched for from one or more donors, or can be obtained from an autologous source. In some embodiments of the aspects described herein, the mesenchymal stem cells are an expanded population of cells.

In some embodiments, the MSCs are administered in combination with the administration of perivascular cells, for example, via systemic IV injection.

Effective amount, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage may vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose may be formulated in animal models to achieve a circulating plasma concentration range that includes the IC50, which achieves a half-maximal inhibition of symptoms as determined in cell culture, or in an appropriate animal model. The effects of any particular dosage can be monitored by a suitable bioassay. The dosage may be determined by a physician and adjusted, as necessary, to suit observed effects of the treatment.

Exemplary modes of administration for use in the methods described herein include, but are not limited to, injection, intrapulmonary (including intranasal and intratracheal) infusion, inhalation (including intranasal), ingestion, and rectal administration. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. The phrases “parenteral administration” and “administered parenterally” as used herein, refer to modes of administration other than enteral and topical administration, usually by injection, and includes, without limitation, intravenous, intraperitoneal, intramuscular, intraarterial, intrathecal, intraventricular, intracapsular, intraorbital, intracardiac, intradermal, transtracheal, subcutaneous, subcuticular, intraarticular, sub capsular, subarachnoid, intraspinal, intracerebro spinal, and intrasternal injection and infusion. In some embodiments of the aspects described herein, one or more routes of administration are used in a subject to achieve distinct effects.

In preferred embodiments, an effective amount of cord tissue-derived mesenchymal stem cells are administered to a subject by a systemic route, e.g., intraperitoneal administration. In some embodiments, the MSCs are injected intraperitoneally into the belly of the subject. In some embodiments, the administration route is intravenous injection. In some embodiments, the administration route is a combination of intraperitoneal and intravenous injections.

The phrases “systemic administration,” “administered systemically”, “peripheral administration” and “administered peripherally” as used herein refer to the administration of a population of mesenchymal stem cells other than directly into a target site, tissue, or organ, such as the lung, such that it enters, instead, the subject's circulatory system and, thus, is subject to metabolism and other like processes.

In some embodiments of the aspects described herein, the methods further comprise administration of one or more therapeutic agents, such as a drug or a molecule, that can enhance or potentiate the effects mediated by the administration of the isolated or enriched mesenchymal stem cells, such as enhancing homing or engraftment of the mesenchymal stem cells, increasing paracrine effects of MSCs, or enhance the survival of the population of MSCs. The therapeutic agent may be a protein (such as an antibody or antigen-binding fragment), a peptide, a polynucleotide, an aptamer, a virus, a small molecule, a chemical compound, a cell, a drug, etc.

In some embodiments, the therapeutic agent is a bispecific antibody. Bispecific antibody (BiAb) technology can combine an effector cell-specific antibody with an injury- or tissue-specific targeting antibody to create a biologic bridge for the purpose of directing cells with reparative or regenerative potential to injured or defective tissue.

“Arming” cells with a therapeutic agent can be performed, for example by incubating the cells with the therapeutic agent, such as a bi-specific antibody. Thus, cells are allowed to bind to the therapeutic agent, such as the antibody specific to the cells. Typically, the cells are thereafter washed to remove unbound therapeutic agents. Thus, as defined herein, “arming” of cells refers to any method wherein a cell for use in the methods described herein is contacted with a therapeutic agent that specifically binds to the cells. In preferred embodiments, the therapeutic agent is specific for the cell and for a molecule expressed on a site to which the cell is to home to. In some embodiments, other homing agents can be used as therapeutic agents and can be similarly bound to the cells by a receptor-ligand interaction.

In some instances, cells can be genetically engineered to express molecules for homing or targeting, such as specific membrane bound receptor molecules or ligands. Such receptors and/or ligands may be engineered to have a cell membrane binding domain and an extracellular domain that will assist in homing of the cells. Methods for genetically engineering cells are well known to one skilled in the art.

Accordingly, in some embodiments, the methods further comprise administration of a antibody or antigen binding fragment for targeting a population of isolated or enriched mesenchymal stem cells being administered using any of the methods described herein to a desired respiratory target tissue in need of repair, for example, the lung alveoli. In some embodiments, the antibody is administered with a population of isolated or enriched mesenchymal stem cells being administered systemically, such as intraperitoneally.

An antibody or antigen-binding fragment for use in such embodiments as a therapeutic agent can be any antibody or antigen-binding fragment specific for an antigen desired to be targeted to using the methods described herein, and can include polyclonal, monoclonal, and bispecific antibodies, and antigen-binding derivatives or fragments thereof. Well-known antigen binding fragments include, for example, single domain antibodies (dAbs; which consist essentially of single VL or VH antibody domains), Fv fragment, including single chain Fv fragment (scFv), Fab fragment, and F(ab′)2 fragment. Methods for the construction of such antibody molecules are well known in the art. In some embodiments of the methods described herein, an antibody or antigen binding fragment is a bispecific antibody. A bispecific antibody refers to an antibody or fragment thereof that can bind to two distinct and unrelated antigens and is generated by combining parts of two separate antibodies that recognize two different antigenic groups. This may be achieved by crosslinking or recombinant techniques. Additionally, moieties may be added to the antibody or a portion thereof to increase half-life in vivo (e.g., by lengthening the time to clearance from the blood stream. Such techniques include, for example, adding PEG moieties (also termed pegylation), and are well-known in the art. See U.S. Patent. Appl. Pub. 20030031671.

An exemplary bispecific antibody for use in arming the cells for the methods described herein is a bispecific antibody that is specific for an antigen on the mesenchymal stem cell (e.g., CD73) and specific for an antigen present on a target tissue.

In some embodiments of the aspects described herein, the methods further comprise administration of one or more surfactants as therapeutic agents, or may be used in combination with one or more surfactant therapies. Surfactant, as used herein, refers to any surface active agent, including but not limited to wetting agents, surface tension depressants, detergents, dispersing agents, emulsifiers. Particularly preferred are those that from a monomolecular layer over pulmonary alveolar surfaces, including but not limited to lipoproteins, lecithins, and sphygomyelins. Exemplary surfactants include, but are not limited to surfactant protein A, surfactant protein B, surfactant protein C, surfactant protein D, and mixtures and combinations thereof. Commercially available surfactants include, but are not limited to, KL-4, Survanta, bLES, Infasurf, Curosurf, HL-10, Alveofact, Surfaxin, Venticute, Pumactant/ALEC, and Exosurf.

The therapeutic methods described herein for the treatment of respiratory or pulmonary conditions using mesenchymal stem cells can be used in conjunction with other therapeutic agents and/or compositions that have been described in detail, see, e.g., Harrison's Principles of Internal Medicine, 15.sup.th edition, 2001, E. Braunwald, et al., editors, McGraw-Hill, New York, N.Y., ISBN 0-07-007272-8, especially chapters 252-265 at pages 1456-1526; Physicians Desk Reference 54.sup.th edition, 2000, pages 303-3251, ISBN 1-56363-330-2, Medical Economics Co., Inc., Montvale, N.J. Treatment of any of these respiratory and pulmonary conditions using a composition may be accomplished using the treatment regimens described herein. For chronic conditions, intermittent dosing can be used to reduce the frequency of treatment. Intermittent dosing protocols are as described herein.

For the clinical use of the methods described herein, isolated or enriched populations of mesenchymal stem cells described herein can be administered along with any pharmaceutically acceptable compound, material, or composition which results in an effective treatment in the subject. Thus, a pharmaceutical formulation for use in the methods described herein can contain an isolated or enriched population of mesenchymal stem cells in combination with one or more pharmaceutically acceptable ingredients.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein and in the claims, the singular forms include the plural reference and vice versa unless the context clearly indicates otherwise. Other than in the operating examples, or where otherwise indicated, all numbers expressing quantities of ingredients or reaction conditions used herein should be understood as modified in all instances by the term “about.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Although any known methods, devices, and materials may be used in the practice or testing of the invention, the methods, devices, and materials in this regard are described herein.

Some embodiments of the invention are listed in the following numbered paragraphs:

paragraph 1. A method for treating or preventing a lung disorder in a subject in need thereof, comprising administering a therapeutically effective amount of a population of isolated or enriched umbilical cord tissue-derived mesenchymal stem cells to said subject via a systemic route.
paragraph 2. The method of paragraph 1, wherein the systemic route is intraperitoneal administration.
paragraph 3. The method of paragraph 1, wherein the systemic route is intravenous injection.
paragraph 4. The method of paragraph 1, wherein the lung disorder is chronic lung disease of the newborn.
paragraph 5. The method of paragraph 1, wherein the subject is an infant or a preterm infant.
paragraph 6. The method of paragraph 1, further comprising selecting a subject who is suffering from a lung disorder prior to administering the population of isolated or enriched umbilical cord tissue-derived mesenchymal stem cells to the subject.
paragraph 7. The method of paragraph 1, wherein the population of isolated or enriched umbilical cord tissue-derived mesenchymal stem cells are expanded or cultured ex vivo prior to administration to the subject.
paragraph 8. The method of paragraph 1, wherein the mesenchymal stem cells are selected based on positive expression of one or more of CD73, CD90, and CD105.
paragraph 9. The method of paragraph 8, wherein the mesenchymal stem cells are selected based on negative expression of one or more of CD34, CD45, CD14, CD19, and HLA-DR.
paragraph 10. The method of paragraph 1, wherein the population of isolated or enriched umbilical cord tissue-derived mesenchymal stem cells are autologous cells.
paragraph 11. The method of paragraph 1, wherein the population of isolated or enriched umbilical cord tissue-derived mesenchymal stem cells are allogeneic cells obtained from one or more donors.
paragraph 12. The method of paragraph 1, further comprising administering at least one therapeutic agent.
paragraph 13. The method of paragraph 12, wherein the at least one therapeutic agent enhances homing, engraftment, or survival of the population of isolated or enriched umbilical cord tissue-derived mesenchymal stem cells.

### DEFINITIONS

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

As used herein the term “comprising” or “comprises” is used in reference to compositions, methods, and respective component(s) thereof, that are useful to an embodiment, yet open to the inclusion of unspecified elements, whether useful or not.

As used herein the term “consisting essentially of” refers to those elements required for a given embodiment. The term permits the presence of elements that do not materially affect the basic and novel or functional characteristic(s) of that embodiment of the invention.

The terms “subject” and “individual” are used interchangeably herein, and refer to an animal, for example, a human from whom cells for use in the methods described herein can be obtained (i.e., donor subject) and/or to whom treatment, including prophylactic treatment, with the cells as described herein, is provided, i.e., recipient subject. For treatment of those conditions or disease states that are specific for a specific animal such as a human subject, the term subject refers to that specific animal. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g. dog, cat, horse, and the like, or production mammal, e.g. cow, sheep, pig, and the like.

Accordingly, for the various embodiments of the methods described herein, a subject is a recipient subject, i.e., a subject to whom the mesenchymal stem cells are being administered, or a donor subject, i.e., a subject from whom a biological sample comprising mesenchymal stem cells are being obtained. A recipient or donor subject can be of any age. In general, the subject can be of any age. In some embodiments, the subject is an adult. In some embodiments, the subject is a “young subject,” defined herein as a subject less than 10 years of age. In other embodiments, the subject is an “infant subject,” defined herein as a subject is less than 2 years of age. In some embodiments, the subject is a “newborn subject,” defined herein as a subject less than 28 days of age. In some embodiments of the aspects described herein, a newborn subject is defined as a subject less than 24 hours of age. A “premature infant subject” or “preterm infant subject” is any subject born before 37 weeks, before 36 weeks, before 35 weeks, before 34 weeks, before 33 weeks, before 32 weeks, before 31 weeks, before 30 weeks, before 29 weeks, before 28 weeks, before 27 weeks, before 26 weeks, before 25 weeks, before 24 weeks, before 23 weeks, before 22 weeks, before 21 weeks, or before 20 weeks of gestation.

As used herein, the terms “administering,” “introducing” and “transplanting” are used interchangeably in the context of the placement of cells, e.g. mesenchymal stem cells, of the invention into a subject, by a method or route which results in at least partial localization of the introduced cells at a desired site, such as a site of injury or repair, such that a desired effect(s) is produced. The cells, e.g. mesenchymal stem cells, can be transplanted directly to the respiratory airways, or alternatively be administered by any appropriate route which results in delivery to a desired location in the subject where at least a portion of the transplanted cells or components of the cells remain viable. The period of viability of the cells after administration to a subject can be as short as a few hours, e.g., twenty-four hours, to a few days, to as long as several years, i.e., long-term engraftment. For example, in some embodiments of the aspects described herein, an effective amount of an isolated or enriched population of mesenchymal stem cells is administered to an infant suffering from bronchopulmonary dysplasia by an intraperitoneal route.

As used herein, the terms “treat,” “treatment,” “treating,” “prevention” or “amelioration” refer to both therapeutic treatment and prophylactic or preventative measures, wherein the object is to prevent, delay the onset, reverse, alleviate, ameliorate, inhibit, or slow down the progression or severity of a condition associated with, a disease or disorder. The term “treating” includes reducing or alleviating at least one adverse effect or symptom of a condition, disease or disorder associated with an inflammatory disease, such as, but not limited to, asthma. Treatment is generally “effective” if one or more symptoms or clinical markers are reduced as that term is defined herein. Alternatively, treatment is “effective” if the progression of a disease is reduced or halted. That is, “treatment” includes not just the improvement of symptoms or markers, but also a cessation or at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable.

The term “treatment” of a disease also includes providing relief from the symptoms or side-effects of the disease (including palliative treatment). For example, any reduction in inflammation, bronchospasm, bronchoconstriction, shortness of breath, wheezing, lower extremity edema, ascites, productive cough, hemoptysis, or cyanosis in a subject suffering from a respiratory disorder, such as asthma, no matter how slight, would be considered an alleviated symptom. In some embodiments of the aspects described herein, the symptoms or a measured parameter of a disease or disorder are alleviated by at least 5%, at least 10%, at least 20%, at least 30%, at least 40%, at least 50%, at least 60%, at least 70%, at least 80%, or at least 90%, upon administration of a population of isolated or enriched for mesenchymal stem cells, as compared to a control or non-treated subject.

Measured or measurable parameters include clinically detectable markers of disease, for example, elevated or depressed levels of a clinical or biological marker, as well as parameters related to a clinically accepted scale of symptoms or markers for a disease or disorder. It will be understood, however, that the total daily usage of the compositions and formulations as disclosed herein will be decided by the attending physician within the scope of sound medical judgment. The exact amount required will vary depending on factors such as the type of disease being treated. “Treatment” can also mean prolonging survival as compared to expected survival if not receiving treatment. Thus, one of skill in the art realizes that a treatment may improve the disease condition, but may not be a complete cure for the disease.

The term “effective amount” as used herein refers to the amount of a population of isolated or enriched for mesenchymal stem cells needed to alleviate at least one or more symptom of the respiratory disease or disorder, and relates to a sufficient amount of pharmacological composition to provide the desired effect, i.e., treat a subject having bronchopulmonary dysplasia. The term “therapeutically effective amount” therefore refers to an amount isolated or enriched for mesenchymal stem cells using the methods as disclosed herein that is sufficient to effect a particular effect when administered to a typical subject, such as one who has or is at risk for bronchopulmonary dysplasia. An effective amount as used herein would also include an amount sufficient to prevent or delay the development of a symptom of the disease, alter the course of a symptom disease (for example but not limited to, slow the progression of a symptom of the disease), or reverse a symptom of the disease. Thus, it is not possible to specify the exact “effective amount”. However, for any given case, an appropriate “effective amount” can be determined by one of ordinary skill in the art using routine experimentation.

The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” as used herein means a pharmaceutically acceptable material, composition or vehicle, such as a liquid or solid filler, diluent, excipient, solvent, media (e.g., stem cell media), encapsulating material, manufacturing aid (e.g., lubricant, talc magnesium, calcium or zinc stearate, or steric acid), or solvent encapsulating material, involved in maintaining the activity of, carrying, or transporting the isolated or enriched populations of mesenchymal stem cells from one organ, or portion of the body, to another organ, or portion of the body.

Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) phosphate buffered solutions; (3) pyrogen-free water; (4) isotonic saline; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) cellulose, and its derivatives, such as sodium carboxymethyl cellulose, methylcellulose, ethyl cellulose, microcrystalline cellulose and cellulose acetate; (17) powdered tragacanth; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (24) C₂-C₁₂ alchols, such as ethanol; (25) starches, such as corn starch and potato starch; and (26) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

As used herein, in vivo (Latin for “within the living”) refers to those methods using a whole, living organism, such as a human subject. As used herein, “ex vivo” (Latin: out of the living) refers to those methods that are performed outside the body of a subject, and refers to those procedures in which an organ, cells, or tissue are taken from a living subject for a procedure, e.g., isolating mesenchymal stem cells from umbilical cord tissue obtained from a donor subject, and then administering the isolated mesenchymal stem cell sample to a recipient subject. As used herein, “in vitro” refers to those methods performed outside of a subject, such as an in vitro cell culture experiment. For example, isolated mesenchymal stem cells can be cultured in vitro to expand or increase the number of mesenchymal stem cells, or to direct differentiation of the mesenchymal stem cells to a specific lineage or cell type, prior to being used or administered according to the methods described herein.

The term “pluripotent” as used herein refers to a cell with the capacity, under different conditions, to differentiate to more than one differentiated cell type, and preferably to differentiate to cell types characteristic of all three germ cell layers. Pluripotent cells are characterized primarily by their ability to differentiate to more than one cell type, preferably to all three germ layers, using, for example, a nude mouse teratoma formation assay. Pluripotency is also evidenced by the expression of embryonic stem (ES) cell markers, although the preferred test for pluripotency is the demonstration of the capacity to differentiate into cells of each of the three germ layers. It should be noted that simply culturing such cells does not, on its own, render them pluripotent. Reprogrammed pluripotent cells (e.g. iPS cells as that term is defined herein) also have the characteristic of the capacity of extended passaging without loss of growth potential, relative to primary cell parents, which generally have capacity for only a limited number of divisions in culture.

The term “progenitor” or “precursor” cell are used interchangeably herein and refer to cells that have a cellular phenotype that is more primitive (i.e., is at an earlier step along a developmental pathway or progression than is a fully differentiated cell) relative to a cell which it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate.

The term “stem cell” as used herein, refers to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential. The term “stem cell” also refers to a subset of progenitors that have the capacity or potential, under particular circumstances, to differentiate to a more specialized or differentiated phenotype, and which retains the capacity, under certain circumstances, to proliferate without substantially differentiating. In one embodiment, the term stem cell refers generally to a naturally occurring mother cell whose descendants (progeny) specialize, often in different directions, by differentiation, e.g., by acquiring completely individual characters, as occurs in progressive diversification of embryonic cells and tissues.

Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a differentiated phenotype, but then “reverse” and re-express the stem cell phenotype, a term often referred to as “dedifferentiation” or “reprogramming” or “retrodifferentiation” by persons of ordinary skill in the art.

The term “adult stem cell” or “ASC” is used to refer to any multipotent stem cell derived from non-embryonic tissue, including fetal, juvenile, and adult tissue. In some embodiments, adult stem cells can be of non-fetal origin. Stem cells have been isolated from a wide variety of adult tissues including blood, bone marrow, brain, olfactory epithelium, skin, pancreas, skeletal muscle, and cardiac muscle. Each of these stem cells can be characterized based on gene expression, factor responsiveness, and morphology in culture. Exemplary adult stem cells include neural stem cells, neural crest stem cells, mesenchymal stem cells, hematopoietic stem cells, and pancreatic stem cells. As indicated above, stem cells have been found resident in virtually every tissue. Accordingly, the present invention appreciates that stem cell populations can be isolated from virtually any animal tissue.

In the context of cell ontogeny, the adjective “differentiated”, or “differentiating” is a relative term meaning a “differentiated cell” is a cell that has progressed further down the developmental pathway than the cell it is being compared with. Thus, stem cells can differentiate to lineage-restricted precursor cells (such as a mesenchymal stem cell), which in turn can differentiate into other types of precursor cells further down the pathway, and then to an end-stage differentiated cell, which plays a characteristic role in a certain tissue type, and may or may not retain the capacity to proliferate further.

The term “differentiated cell” is meant any primary cell that is not, in its native form, pluripotent as that term is defined herein. Stated another way, the term “differentiated cell” refers to a cell of a more specialized cell type derived from a cell of a less specialized cell type (e.g., a stem cell such as a mesenchymal stem cell) in a cellular differentiation process.

As used herein, the term “somatic cell” refers to are any cells forming the body of an organism, as opposed to germline cells. In mammals, germline cells (also known as “gametes”) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated stem cells—is a somatic cell: internal organs, skin, bones, blood, and connective tissue are all made up of somatic cells. In some embodiments the somatic cell is a “non-embryonic somatic cell”, by which is meant a somatic cell that is not present in or obtained from an embryo and does not result from proliferation of such a cell in vitro. In some embodiments the somatic cell is an “adult somatic cell”, by which is meant a cell that is present in or obtained from an organism other than an embryo or a fetus or results from proliferation of such a cell in vitro.

As used herein, the term “adult cell” refers to a cell found throughout the body after embryonic development.

The term “phenotype” refers to one or a number of total biological characteristics that define the cell or organism under a particular set of environmental conditions and factors, regardless of the actual genotype.

The term “cell culture medium” (also referred to herein as a “culture medium” or “medium”) as referred to herein is a medium for culturing cells containing nutrients that maintain cell viability and support proliferation. The cell culture medium may contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art.

The term “cell line” refers to a population of largely or substantially identical cells that has typically been derived from a single ancestor cell or from a defined and/or substantially identical population of ancestor cells. The cell line may have been or may be capable of being maintained in culture for an extended period (e.g., months, years, for an unlimited period of time). It may have undergone a spontaneous or induced process of transformation conferring an unlimited culture lifespan on the cells. Cell lines include all those cell lines recognized in the art as such. It will be appreciated that cells acquire mutations and possibly epigenetic changes over time such that at least some properties of individual cells of a cell line may differ with respect to each other.

The terms “renewal” or “self-renewal” or “proliferation” are used interchangeably herein, are used to refer to the ability of stem cells to renew themselves by dividing into the same non-specialized cell type over long periods, and/or many months to years. In some instances, proliferation refers to the expansion of cells by the repeated division of single cells into two identical daughter cells.

The term “lineages” is used herein describes a cell with a common ancestry or cells with a common developmental fate. In the context of a cell that is of mesenchymal origin or is “mesenchymal linage” this means the cell was derived from a mesenchymal stem cell and can differentiate along lineage restricted pathways, such as one or more developmental lineage pathways which give rise to mesenchymal cells, which in turn can differentiate into other cell types.

As used herein, the term “xenogeneic” refers to cells that are derived from different species.

The term “isolated cell” as used herein refers to a cell that has been removed from an organism in which it was originally found or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched from.

The term “modulate” is used consistently with its use in the art, i.e., meaning to cause or facilitate a qualitative or quantitative change, alteration, or modification in a process, pathway, or phenomenon of interest. Without limitation, such change may be an increase, decrease, or change in relative strength or activity of different components or branches of the process, pathway, or phenomenon. A “modulator” is an agent that causes or facilitates a qualitative or quantitative change, alteration, or modification in a process, pathway, or phenomenon of interest.

The term “tissue” refers to a group or layer of specialized cells which together perform certain special functions. The term “tissue-specific” refers to a source of cells from a specific tissue.

The term “statistically significant” or “significantly” refers to statistical significance and generally means a two standard deviation (2SD) difference.

Other than in the operating examples, or where otherwise indicated, all numbers expressing quantities of ingredients or reaction conditions used herein should be understood as modified in all instances by the term “about.” The term “about” when used in connection with percentages may mean±5% of the value being referred to. For example, about 100 means from 95 to 105.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### EXAMPLES

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

The technology described herein is further illustrated by the following examples which in no way should be construed as being further limiting.

### Example 1

### Intranasal Versus Intraperitoneal Delivery of Human Umbilical Cord Tissue-Derived Cultured Mesenchymal Stromal Cells in a Murine Model of Neonatal Lung Injury

The morphologic and functional effects of intranasal (IN) versus intraperitoneal (IP) MSC administration were studied in a rodent model of neonatal lung injury. Cultured human cord tissue MSCs (0.1, 0.5 or 1×10⁶ cells/pup) were given IN or IP to newborn SCID-beige mice exposed to 90% O₂ from birth; sham controls received equal volume PBS. Lung mechanics, engraftment, lung growth and alveolarization were evaluated 8 weeks post-transplantation. High-dose IP MSC administration to newborn mice exposed to 90% O₂ resulted in restoration of normal lung compliance, elastance and pressure-volume loops (tissue recoil). Histologically, high-dose IP MSC administration was associated with alveolar septal widening, suggestive of interstitial matrix modification. IN MSC or lower-dose IP administration had no significant effects on lung function or alveolar remodeling. Pulmonary engraftment was rare in all groups. The findings suggest that high dose systemic administration of human cultured MSCs can restore normal compliance in neonatally injured lungs, possibly by paracrine modulation of the interstitial matrix. Intranasal delivery had no obvious pulmonary effects.

### Materials and Methods

Isolation, Culture, and Characterization of Cord Mesenchymal Stromal Cells

Human cultured umbilical cord tissue-derived MSCs (TC-MSC, further described as MSC) were used in all experiments. Umbilical cord tissue was procured from uncomplicated full-term deliveries at The Christ Hospital (Cincinnati, Ohio), according to protocols approved by the hospital's Institutional Review Board, and sent to the Viacord Processing Lab (Cincinnati, Ohio). Upon receipt, the cord was cleaned with a chlorhexadine wipe. The cord was placed into a sterile cup with 10 mL of antibiotic solution (25 μg/mL gentamycin, 100 IU/mL penicillin, 100 μg/mL streptomycin, 0.25 μg/mL Amphotericin B, all from Lonza, Basel, CH). Following rinses with sterile PBS, the cord tissue underwent overnight digestion in collagenase (Collagenase NB 6, GMP grade, 0.75 mg/ml, Serva, Heidelberg, Del.) with antibiotics in a CaCl₂-buffered digestion solution (37° C.). The homogenate was centrifuged to pellet the cell suspension, washed several times and resuspended in DMSO freezing media.

Frozen cell aliquots were thawed at 37° C. and resuspended in culture media (DMEM supplemented with 20% FBS (both from StemCell Technologies, Vancouver, BC), 1% penicillin/streptomycin and 1% L-glutamine (Lonza). Cells were cultured on collagen-coated plates (37° C., 5% CO₂); medium was replaced every 3-4 days. Upon reaching 70-80% confluence, MSCs were trypsinized (0.25% Trypsin-EDTA, Life Technologies, Carlsbad, Calif.) to a new passage.

Cultured MSCs at passages 4-10 were used in all experiments. The cells were surface stained using a panel of flow cytometry anti-human antibodies against CD73, CD90, CD105, CD34, CD45, CD14, HLA-ABC, CD49c, CD49e, HLA-DR (Biosciences-BD Pharmingen, San Jose, Calif.), CD49d and CD49f (eBioscience, San Diego, Calif.), and analyzed by flow cytometry. The MSC line selected for this study expressed the mesenchymal stem cell markers CD73, CD90, and CD105. In addition, the cells also expressed HLA class I and various cell adhesion markers (CD49c, CD49d, CD49e, and CD49f. The cells were negative for hematopoietic cell surface antigens, CD34, CD45, CD14, CD19 and HLA-DR (HLA class II, not shown). These molecular characteristics conform to the consensus criteria for defining (human) mesenchymal stromal cells established by The International Society for Cellular Therapy (Dominici M, et al.: Minimal criteria for defining multipotent mesenchymal stromal cells. The International Society for Cellular Therapy position statement, Cytotherapy 2006, 8:315-317).

Animal Husbandry, Hyperoxia Exposure, and Cell Administration

Six-week-old timed pregnant SCID-beige mice (Fox Chase SCID beige; T- and B-cell deficient; NK cell-impaired) were obtained from Charles River laboratories (Wilmington, Mass.) and maintained under pathogen-free conditions. Newborn mice were exposed to room air or hyperoxia (90% O₂) from birth until postnatal day 7 (P7; day of birth=P1). For hyperoxia exposure, mice were placed in an airtight Plexiglass chamber. Oxygen concentrations were continuously monitored and controlled with an in-line oxygen analyzer and controller system (ProOx 110; BioSpherix, Redfield, N.Y.). Nursing dams were rotated daily between air- and oxygen-exposed litters to minimize maternal oxygen toxicity.

At P5, corresponding to a time point of intense acute lung injury and active tissue remodeling, the pups were randomly assigned to MSC administration given by intranasal (IN) or intraperitoneal (IP) route. For IN inoculation, 20 μl of cell suspension, containing 0.1, 0.5, or 1×10⁶ cells, was placed over the nasal orifices, as previously described (Fritzell J A, Jr., et al.: Fate and effects of adult bone marrow cells in lungs of normoxic and hyperoxic newborn mice, Am J Respir Cell Mol Biol 2009, 40:575-587), thus ensuring aspiration of stem cells into the lungs. For IP delivery, a 25 μL Hamilton syringe (Hamilton, Reno, Nev.) with 26 gauge needle was used for injection of the cell suspension (0.1, 0.5, or 1×10⁶ cells in 20 μl PBS) in the left lower quadrant. The injection was preceded by aspiration to ensure proper localization of the needle. Hyperoxia-exposed sham controls received equal-volumes of vehicle buffer (PBS). The IN and IP deliveries were well tolerated by both normoxic and hyperoxia-exposed pups. The animals were sacrificed at 48 hours or 8 weeks post-transplantation. All animal experiments were approved by the institutional animal care and use committee (IACUC) and conducted in accordance with institutional guidelines for the care and use of laboratory animals.

Analysis of Lung Mechanics (FlexiVent)

Invasive lung function testing was performed at 8 weeks post-transplantation by the forced oscillation technique in anesthetized, non-paralyzed, tracheotomized animals with intact chest wall (Vanoirbeek J A, et al.: Noninvasive and invasive pulmonary function in mouse models of obstructive and restrictive respiratory diseases, Am J Respir Cell Mol Biol 2010, 42:96-104). Mice were deeply anesthetized with an IP injection of ketamine (140 mg/kg) and xylazine (14 mg/kg) to eliminate all spontaneous breathing under anesthesia. Body weights were recorded at the start of the procedure. The tracheal cannula was connected to a computer-controlled small animal ventilator (FlexiVent, SCIREQ, Montreal, PQ, Canada). The mice were ventilated with a tidal volume of 10 mL/kg at an average breathing frequency of 150 breaths/min and a positive end-expiratory pressure (PEEP) of 3 cm H₂O to prevent alveolar collapse. Lung function parameters were calculated by fitting pressure and volume data to the single compartment and constant phase models (Bates J: Lung mechanics. Edited by New York, Cambridge University Press, 2009). Resistance (Rrs), compliance (Crs), and elastance (Ers) of the entire respiratory system (airways, lungs, and chest wall) were measured with the Snapshot-150 v7.0 perturbation. Rn (Newtonian resistance, a measure of central airway resistance), tissue damping (resistance, G), and tissue elastance (H) were measured with the subsequent Quickprime-3 v7.0 forced oscillation perturbation. Tissue hysteresivity (G/H, eta) was calculated from G and H values. Finally, maximal PV loops were generated between +30 cm H₂O and −30 cm H₂O pressure (PVr-P=PV-ramp pressure regulated) to obtain maximal vital (total) lung capacity (A), inspiratory capacity (IC) from zero pressure (B), form of deflating PV loop (K), quasi-static compliance (Cst) and elastance (Est), and hysteresis (area between inflating and deflating part of the PV loop). All maneuvers and perturbations were performed until at least three reproducible measurements were recorded. A coefficient of determination of 0.95 was the lower limit for acceptance of a measurement. For each parameter and each animal, the average of at least three measurements was calculated. The individuals performing the lung function studies and those analyzing the data were blinded with respect to the experimental group of the animals. All data were collected using the FlexiVent FX software with FlexiWare 7.0 and analyzed offline using Excel (Microsoft, Redmond, Wash.).

Tissue Processing and Bronchoalveolar Lavage

Following lung function testing, the animals were euthanized by sectioning of diaphragm and abdominal vessels. Bronchoalveolar lavage was performed by repetitive tracheal instillation and aspiration of 0.7 ml sterile saline (0.9% NaCl) until diffuse and complete expansion of the parenchyma was observed. The recovered fluid was pooled. Cells were spun onto microscope slides (1,500 rpm, 5 min) (Shannon Cytospin-4 cytocentrifuge, Thermo Scientific, Waltham, Mass.), air-dried, and Giemsa-Wright stained for differential cell counts of macrophages, eosinophils, neutrophils, and lymphocytes. Some slides were Perls-stained for detection of iron pigment.

Following lavage, the lungs were formalin-fixed by standardized tracheal instillation at a constant pressure of 20 cm H₂O. All lungs were inflated equally on the same apparatus. Immediately after inflation, the trachea was ligated and the lungs were immersed in formalin for overnight fixation. Selected organs (liver, kidneys, spleen and heart) were resected and immersion-fixed in formalin. In order to examine the distribution of transplanted cells, selected animals were studied 48 hours after cell administration. In these animals, all abdominal organs and tissues were removed and immersion-fixed en bloc. After overnight fixation, the tissues were dehydrated in graded ethanol solutions, embedded in paraffin, and stained with hematoxylin-eosin.

Analysis of Cell Fate and Engraftment

The presence and localization of MSCs following intranasal or intraperitoneal administration was monitored by taking advantage of the species mismatch between the human cord blood-derived MSCs and their murine host. Systemic and pulmonary distribution of MSCs was tracked by anti-vimentin immunohistochemical analysis, using a specific anti-human vimentin antibody (N1521, DAKO, Glostrup, Denmark). This antibody does not recognize the mouse antigen. Antibody binding was detected by streptavidin-biotin immunoperoxidase method. In addition, the presence of MSCs or their progeny was studied by fluorescent in situ hybridization (FISH) analysis using human-specific alu probes (PR-1001-01, BioGenex, San Ramon, Calif.), as previously described (De Paepe M E, et al.: Alveolar epithelial cell therapy with human cord blood-derived hematopoietic progenitor cells, Am J Pathol 2011, 178:1329-1339). The proliferative activity of engrafted cord blood-derived cells was assessed by combining human alu-FISH analysis with anti-Ki67 immunohistochemistry, as previously described (De Paepe M E, et al.: Alveolar epithelial cell therapy with human cord blood-derived hematopoietic progenitor cells, Am J Pathol 2011, 178:1329-1339). In addition to standard epifluorescence microscopy, the sections were viewed by confocal microscopy. Slice or three-dimensional volume reconstruction and projections were generated to ascertain the veracity of co-localization phenomena, as previously described (De Paepe M E, et al.: Alveolar epithelial cell therapy with human cord blood-derived hematopoietic progenitor cells, Am J Pathol 2011, 178:1329-1339; Mao Q, et al.: Ex vivo expanded human cord blood derived hematopoietic progenitor cells induce lung growth and alveolarization in injured newborn lungs, Respir Res 2013, 14:37; Fritzell J A, Jr., et al.: Fate and effects of adult bone marrow cells in lungs of normoxic and hyperoxic newborn mice, Am J Respir Cell Mol Biol 2009, 40:575-587).

Histomorphometric Analysis of Lung Growth and Alveolar Remodeling

Morphometric assessment of the growth of peripheral air-exchanging lung parenchyma and contribution of the various lung compartments (airspace versus parenchyma) to the total lung volume was performed using stereological volumetric techniques, as previously described (De Paepe M E, et al.: Lung growth response after tracheal occlusion in fetal rabbits is gestational age-dependent, Am J Respir Cell Mol Biol 1999, 21:65-76; De Paepe M E, et al.: Temporal pattern of accelerated lung growth after tracheal occlusion in the fetal rabbit, Am J Pathol 1998, 152:179-190). The inflated lung volume, V(lu), was determined according to the Archimedes principle (Aherne W A, Dunnill, M. S.: The estimation of whole organ volume. Edited by Aherne W A, Dunnill, M. S. London, Edward Arnold Ltd., 1982, p. pp. 10-18). The areal density of air-exchanging parenchyma, AA(ae/lu), was determined by point-counting based on computer-assisted image analysis. The total volume of air-exchanging parenchyma, V(ae), was calculated by multiplying AA(ae/lu) by V(lu). Alveolarization was quantified by computer-assisted histomorphometric analysis of the mean cord length (MCL) and mean septal wall thickness, as previously described (De Paepe M E, et al.: Fas-ligand-induced apoptosis of respiratory epithelial cells causes disruption of postcanalicular alveolar development, Am J Pathol 2008, 173:42-56). All morphometric assessments were made on coded slides by a single observer who was unaware of the experimental condition of the animal analyzed.

Data Analysis

Values are expressed as mean±standard deviation (SD) or standard error of mean (SEM). Statistical analyses were performed using standard one-way ANOVA with Dunnett's multiple comparison test (GraphPad Prism; GraphPad Software, Inc., San Diego, Calif.). The significance level was set at P<0.05.

### Results

Effects of MSC Administration to Hyperoxic Newborn Mice on Somatic Growth, Lung Growth, and Alveolar Remodeling

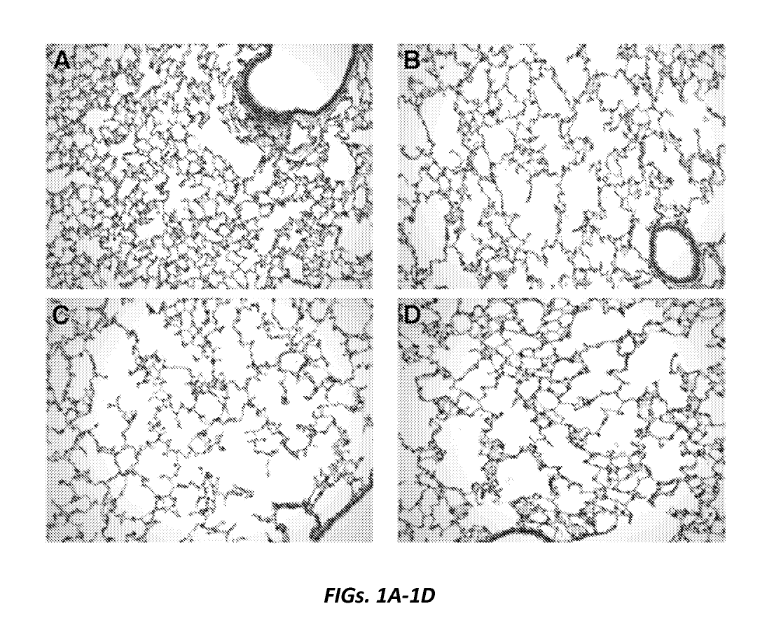

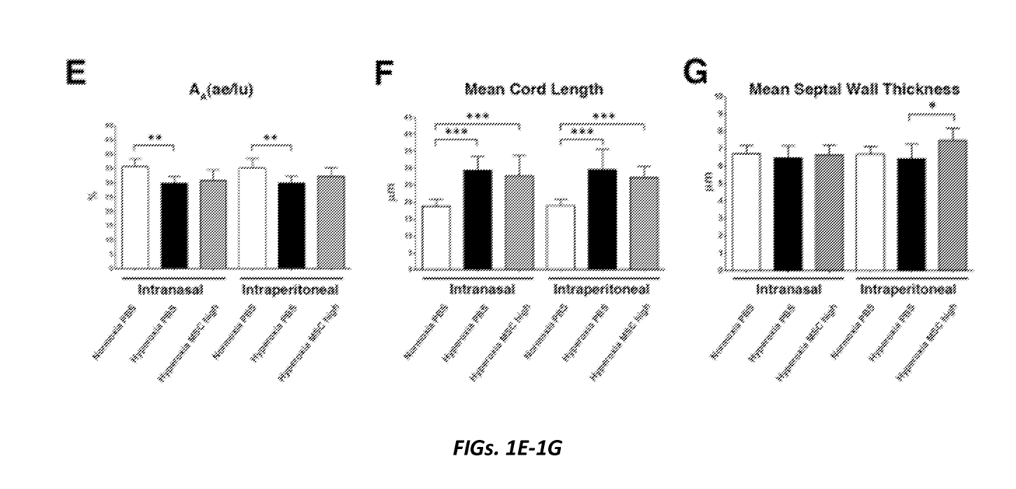

Newborn mice were exposed to 90% O₂ from birth until P7, treated with MSC (IN or IP) on P5, and sacrificed 8 weeks post-transplantation. The long-term effects of neonatal hyperoxia exposure on somatic and lung growth were determined by comparative analysis of PBS-treated normoxic and hyperoxic controls (Tables 1 and 2). Hyperoxia during the first neonatal week (late saccular to early alveolar stage of development) had a prolonged adverse impact on somatic growth, resulting in a 23% and 14% reduction in body weight in intranasal and intraperitoneal PBS-treated control groups, respectively. As expected, histopathologic examination of the lungs of hyperoxia-exposed control animals revealed expanded, simplified airspaces, contrasting with the complex alveolar network of smaller, polygonal airspaces seen in normoxic controls (FIGS. 1A-1B). Stereologic volumetry demonstrated a significant reduction in areal density of air-exchanging parenchyma (AA(ae/lu)) and volume of air-exchanging parenchyma (V(ae)) in hyperoxic versus normoxic control animals, while the ratio of V(ae) over body weight (V(ae)/BW) remained equivalent between both groups (FIG. 1E and Tables 1 and 2). In agreement with their emphysema-like lung morphology, the mean cord length of hyperoxic controls was significantly 56% larger than that of normoxic controls, reflective of diminished alveolar septation (alveolar simplification) (FIG. 1F). The mean septal wall thickness of hyperoxic controls was slightly smaller than that of normoxic controls (difference not significant) (Tables 1 and 2) (FIG. 1G).

TABLE 1
Biometry and lung morphometry (90% O₂ experiment). Intranasal administration.

| | NORMOXIA | HYPEROXIA | | | |
|---|---|---|---|---|---|
| | PBS (22) | PBS (10) | MSC LOW (7) | MSC MEDIUM (6) | MSC HIGH (7) |
| Body wt (g) | 21.15 ± 3.38 | 16.20 ± 2.58*** | 17.61 ± 1.49 | 17.12 ± 2.52 | 16.61 ± 2.78* |
| Lung wt (infl) (mg) | 508 ± 68 | 447 ± 87 | 461 ± 79 | 475 ± 54 | 455 ± 72 |
| Heart wt (mg) | 160 ± 30 | 129 ± 23 | 146 ± 16 | 138 ± 35 | 135 ± 29 |
| Heart wt/BW (%) | 0.75 ± 0.08 | 0.79 ± 0.06 | 0.83 ± 0.07 | 0.80 ± 0.11 | 0.81 ± 0.09 |
| V(lung) (μl) | 482 ± 65 | 424 ± 82 | 438 ± 75 | 451 ± 52 | 433 ± 69 |
| V(lung)/BW (μl/g) | 23.05 ± 2.92 | 26.13 ± 2.62 | 24.83 ± 3.37 | 26.47 ± 1.03 | 26.19 ± 2.98 |
| A_{A}(ae/lu) (%) | 35.59 ± 2.87 | 29.99 ± 2.19** | 31.44 ± 4.32 | 30.72 ± 3.31 | 30.80 ± 3.73 |
| V(ae) (μl) | 168 ± 11 | 127 ± 28*** | 144 ± 33 | 138 ± 16 | 136 ± 34 |
| V(ae)/BW (μl/g) | 7.65 ± 0.75 | 7.83 ± 0.93 | 7.97 ± 1.64 | 8.15 ± 1.10 | 8.04 ± 1.86 |
| MCL (μm) | 18.82 ± 2.07 | 29.35 ± 4.07**** | 28.56 ± 4.58**** | 28.52 ± 2.31**** | 27.81 ± 5.93**** |
| MSWT (μm) | 6.71 ± 0.49 | 6.48 ± 0.70 | 6.53 ± 0.41 | 6.45 ± 0.65 | 6.67 ± 0.57 |

In Table 1, Values represent mean±SD of (n) animals per group. Experimental animals were treated with 0.1×10⁶ (MSC low), 0.5×10⁶ (MSC medium) or 1×10⁶ (MSC high) mesenchymal stromal cells via intranasal route. MSC: expanded mesenchymal stromal cells; BW: body weight; AA(ae/lu): areal density of air-exchanging parenchyma; V(ae): volume of air-exchanging parenchyma; MCL: mean cord length; MSWT: mean septal wall thickness. *: P<0.05; **: P<0.01; ***: P<0.001; ****: P<0.0001 versus PBS IN normoxia.

TABLE 2
Biometry and lung morphometry (90% O₂ experiment). Intraperitoneal administration.

| | NORMOXIA | HYPEROXIA | | | |
|---|---|---|---|---|---|
| | PBS (21) | PBS (11) | MSC LOW (7) | MSC MEDIUM (7) | MSC HIGH (7) |
| Body wt (g) | 21.04 ± 3.19 | 18.08 ± 2.21 | 16.21 ± 2.14** | 16.41 ± 3.20* | 15.80 ± 2.78** |
| Lung wt (infl) (mg) | 503 ± 51 | 495 ± 53 | 408 ± 57 | 449 ± 86 | 431 ± 64 |
| Heart wt (mg) | 161 ± 29 | 141 ± 21 | 128 ± 23 | 132 ± 14 | 127 ± 28 |
| Heart wt/BW (%) | 0.77 ± 0.10 | 0.78 ± 0.09 | 0.79 ± 0.10 | 0.81 ± 0.09 | 0.81 ± 0.12 |
| V(lung) (μl) | 478 ± 48 | 471 ± 50 | 388 ± 54 | 426 ± 82 | 409 ± 60 |
| V(lung)/BW (μl/g) | 22.99 ± 2.36 | 26.35 ± 4.10 | 25.11 ± 3.61 | 26.06 ± 2.91 | 26.04 ± 1.30 |
| A_{A}(ae/lu) (%) | 35.11 ± 3.38 | 29.98 ± 2.46** | 32.27 ± 3.98 | 32.44 ± 2.18 | 32.25 ± 3.10 |
| V(ae) (μl) | 171 ± 23 | 141 ± 16 | 125 ± 18** | 141 ± 28 | 133 ± 33* |
| V(ae/BW) (μl/g) | 8.00 ± 1.24 | 7.89 ± 1.31 | 7.99 ± 0.80 | 8.52 ± 1.15 | 8.47 ± 0.88 |
| MCL (μm) | 18.92 ± 1.92 | 29.45 ± 6.24**** | 27.32 ± 3.64*** | 27.94 ± 5.11**** | 27.18 ± 3.40*** |
| MSWT (μm) | 6.70 ± 0.46 | 6.44 ± 0.83 | 6.56 ± 0.71 | 6.72 ± 0.58 | 7.48 ± 0.71° |

In Table 2, values represent mean±SD of (n) animals per group. Experimental animals were treated with 0.1×10⁶ (MSC low), 0.5×10⁶ (MSC medium) or 1×10⁶ (MSC high) mesenchymal stromal cells via intraperitoneal route. MSC: expanded mesenchymal stromal cells; BW: body weight; AA(ae/lu): areal density of air-exchanging parenchyma; V(ae): volume of air-exchanging parenchyma; MCL: mean cord length; MSWT: mean septal wall thickness. *: P<0.05; **: P<0.01; ***: P<0.001; ****: P<0.0001 versus PBS IP normoxia. °: P<0.05 versus PBS IP hyperoxia.

After establishment of baseline values in normoxic and hyperoxic PBS-treated control animals, the effects of intraperitoneal or intranasal administration of MSCs (low dose: 0.1×10⁶; medium dose: 0.5×10⁶; high dose: 1×10⁶) on somatic and lung growth were determined 8 weeks after transplantation. Intranasal MSC administration had no significant effects on body weight or lung growth (Table 1). Similarly, intranasal MSCs had no effect on alveolar remodeling, as assessed by light microscopy (FIG. 1C), AA(ae/lu), mean cord length, and mean septal wall thickness (FIGS. 1E-1G).

Intraperitoneal MSC administration tended to be associated with a further reduction in body weight in hyperoxic animals (body weight: 15.80±2.78 g in high-dose MSC group versus 18.08±2.21 g in hyperoxic controls, difference not significant) (Table 2). The morphology of IP MSC-treated lungs appeared similar to that of hyperoxia-exposed controls by light microscopic inspection (FIG. 1D). The airspaces were enlarged with obvious diminished alveolar septation compared with normoxic controls. Computer-assisted morphometric analysis revealed several tendencies in the intraperitoneal MSC treatment group, specifically: a relative increase in areal density of air-exchanging parenchyma (AA(ae/lu): 32.25±3.10% in high-dose MSC group versus 29.98±2.46% in hyperoxic controls); a relative increase in V(ae)/BW (8.47±0.88 μl/g in high-dose MSC group versus 7.89±1.31 μl/g in hyperoxic controls); and a mild decrease in MCL (27.18±3.40 μm in high-dose MSC group versus 29.45±6.24 μm in hyperoxic controls) (Table 2) (FIGS. 1E-1F). Interestingly, the mean septal wall thickness of MSC-treated hyperoxic animals (high dose) was significantly larger than that of hyperoxic control animals (7.48±0.71 μm in high-dose MSC group versus 6.44±0.83 in hyperoxic controls, P<0.05) (FIG. 1G), consistent with MSC-related modification of the pulmonary interstitium.

Functional Effects of MSC Administration to Hyperoxic Newborn Mice

It was observed that intraperitoneal MSC administration to hyperoxic newborn pups was associated with significant alveolar septal widening and modest alteration of other morphometric outcome parameters of alveolarization, such as mean cord length and AA(ae/lu). Intranasal MSC administration had no morphologic or morphometric effects. To determine whether MSC administration had any lasting functional effects, lung mechanics were studied by forced oscillation technique (FlexiVent), 8 weeks after intraperitoneal or intranasal administration of MSCs (low, medium, or high dose) or PBS. The lung mechanics of PBS-treated normoxic controls was first compared with those of hyperoxia-exposed controls to determine whether neonatal exposure to 90% O₂ has any long-term functional effects. As shown in Tables 3 and 4, this severe neonatal hyperoxia regimen resulted in significantly increased pulmonary compliance (compliance of the respiratory system, Crs; as well as quasi-static compliance, Cst), reduced elastance (elastance of the respiratory system, Ers; as well as tissue elastance, H), increased inspiratory capacity, increased hysteresivity (eta), increased total lung capacity (Salazar-Knowles parameter A) and increased curvature of the upper portion of the deflation PV curve (Salazar-Knowles parameter K) in adulthood. In contrast, neonatal hyperoxia exposure had no lasting effects on central airway resistance (Newtonian resistance, Rn), resistance of the respiratory system (Rrs), or area of the pressure-volume (PV) loop (Tables 3 and 4).

TABLE 3
Mechanical lung function parameters (90% O₂ experiment). Intranasal administration.

| | NORMOXIA | HYPEROXIA | | | |
|---|---|---|---|---|---|
| | PBS (22) | PBS (10) | MSC LOW (7) | MSC MEDIUM (6) | MSC HIGH (7) |
| Inspiratory | 0.70 | 0.77 | 0.85* | 0.80 | 0.79 |
| capacity | (0.56-0.87) | (0.64-1.13) | (0.79-0.96) | (0.64-0.97) | (0.71-0.88) |
| Rrs | 0.57 | 0.59 | 0.53 | 0.55 | 0.56 |
| | (0.4-0.69) | (0.49-0.86) | (0.50-0.79) | (0.49-0.69) | (0.51-0.69) |
| Crs | 0.043 | 0.054** | 0.055* | 0.054 | 0.053* |
| | (0.033-0.055) | (0.046-0.072) | (0.051-0.063) | (0.041-0.064) | (0.044-0.065) |
| Ers | 23.40 | 18.54** | 18.24** | 18.63* | 18.96* |
| | (18.29-29.98) | (14.04-21.08) | (15.85-19.61) | (15.55-24.38) | (15.31-22.66) |
| Rn | 0.289 | 0.284 | 0.277 | 0.277 | 0.296 |
| | (0.219-0.328) | (0.251-0.560) | (0.235-0.349) | (0.219-0.343) | (0.225-0.336) |
| G | 3.97 | 3.87 | 3.67 | 3.55 | 3.47 |
| | (3.38-4.98) | (3.13-4.51) | (3.23-4.45) | (3.30-4.22) | (3.28-5.01) |
| H | 22.26 | 15.15**** | 15.63**** | 17.05*** | 16.23**** |
| | (19.46-27.40) | (10.56-18.29) | (13.01-17.56) | (14.22-21.35) | (12.23-18.91) |
| eta (G/H) | 0.17 | 0.24**** | 0.23**** | 0.22* | 0.24*** |
| | (0.15-0.22) | (0.21-0.30) | (0.20-0.34) | (0.19-0.24) | (0.19-0.27) |
| Cst | 0.070 | 0.079 | 0.090** | 0.088 | 0.084 |
| | (0.055-0.094) | (0.064-0.116) | (0.083-0.104) | (0.069-0.103) | (0.076-0.102) |
| A | 0.707 | 0.755 | 0.845* | 0.785 | 0.774 |
| | (0.559-0.855) | (0.626-1.092) | (0.776-0.936) | (0.635-0.945) | (0.700-0.941) |
| K | 0.167 | 0.187**** | 0.1885**** | 0.190**** | 0.190**** |
| | (0.151-0.186) | (0.176-0.199) | (0.177-0.198) | (0.179-0.207) | (0.185-0.196) |
| Area | 1.63 | 1.54 | 1.94 | 1.67 | 1.70 |
| | (1.12-2.25) | (1.26-2.25) | (1.76-2.14) | (1.36-2.11) | (1.32-2.15) |

In Table 3, values represent median (minimum-maximum) of (N) animals per group. Experimental animals were treated with 0.1×10⁶ (MSC low), 0.5×10⁶ (MSC medium) or 1×10⁶ (MSC high) mesenchymal stromal cells via intranasal route. *: P<0.05; **: P<0.01; ***: P<0.001; ****: P<0.0001 versus PBS IN normoxia.

TABLE 4
Mechanical lung function parameters (90% O₂ experiment). Intraperitoneal administration.

| | NORMOXIA | HYPEROXIA | | | |
|---|---|---|---|---|---|
| | PBS (21) | PBS (11) | MSC LOW (7) | MSC MEDIUM (7) | MSC HIGH (7) |
| Inspiratory | 0.71 | 0.87** | 0.73 | 0.81 | 0.62§ |
| capacity | (0.55-0.82) | (0.70-0.98) | (0.66-1.03) | (0.67-1.05) | (0.56-0.96) |
| Rrs | 0.57 | 0.57 | 0.57 | 0.54 | 0.62 |
| | (0.51-0.80) | (0.49-0.71) | (0.48-0.66) | (0.49-0.72) | (0.49-1.02) |
| Crs | 0.044 | 0.059**** | 0.048 | 0.059 | 0.044§§ |
| | (0.034-0.051) | (0.045-0.072) | (0.042-0.068) | (0.943-0.072) | (0.033-0.064) |
| Ers | 22.97 | 17.05**** | 20.91 | 20.05 | 23.18§§ |
| | (19.72-29.85) | (13.87-22.18) | (14.68-23.62) | (13.94-23.44) | (15.56-30.23) |
| Rn | 0.271 | 0.288 | 0.280 | 0.288 | 0.279 |
| | (0.223-0.494) | (0.224-0.400) | (0.251-0.337) | (0.238-0.372) | (0.244-0.341) |
| G | 3.84 | 3.54 | 3.99 | 3.58 | 4.29*§§ |
| | (3.20-4.98) | (2.69-4.23) | (2.85-5.03) | (2.77-5.11) | (3.26-6.77) |
| H | 22.36 | 13.97**** | 18.27** | 18.09*** | 20.37§§ |
| | (19.33-28.13) | (12.15-19.40) | (13.44-20.69) | (13.25-20.04) | (13.49-24.74) |
| eta (G/H) | 0.175 | 0.23**** | 0.22** | 0.215* | 0.23**** |
| | (0.14-0.19) | (0.22-0.30) | (0.19-0.26) | (0.17-0.27) | (0.20-0.32) |
| Cst | 0.071 | 0.088** | 0.078 | 0.087* | 0.067 |
| | (0.056-0.081) | (0.073-0.104) | (0.070-0.110) | (0.073-0.116) | (0.053-0.102) |
| A | 0.711 | 0.844* | 0.721 | 0.794 | 0.612§ |
| | (0.558-0.812) | (0.688-0.963) | (0.655-0.997) | (0.660-1.026) | (0.543-0.937) |
| K | 0.1645 | 0.184**** | 0.191**** | 0.1875**** | 0.1815* |
| | (0.150-0.179) | (0.176-0.198) | (0.180-0.198) | (0.178-0.205) | (0.148-0.197) |
| Area | 1.72 | 1.84 | 1.60 | 1.60 | 1.22§ |
| | (1.14-2.20) | (1.58-2.22) | (1.36-2.22) | (1.44-2.53) | (1.09-1.97) |

In Table 4, values represent median (minimum-maximum) of (N) animals per group. Experimental animals were treated with 0.1×10⁶ (MSC low), 0.5×10⁶ (MSC medium) or 1×10⁶ (MSC high) mesenchymal stromal cells via intraperitoneal route. *: P<0.05; **: P<0.01; ***: P<0.001; ****: P<0.0001 versus PBS IP normoxia. §: P<0.05; §§: P<0.01 versus PBS IP hyperoxia.

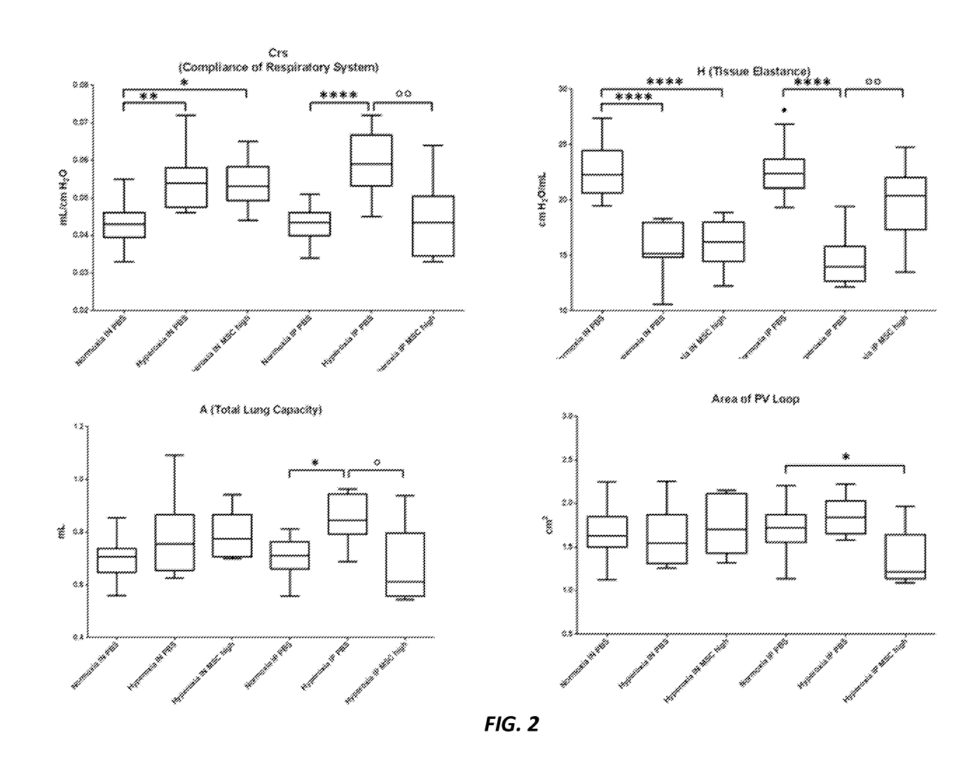

After determination of the baseline lung mechanics of hyperoxia-exposed control animals, the long-term functional effects of neonatal MSC administration was studied. Intranasal MSC administration had no obvious effects on any of the lung function parameters studied, specifically: the pulmonary compliance, elastance, hysteresivity and inspiratory capacity of animals treated with intranasal MSCs were similar to those of hyperoxic PBS-treated controls, regardless of the MSC dose used (Table 3). In sharp contrast, intraperitoneal MSC administration had a significant and seemingly dose-dependent impact on several lung function parameters. At the highest dose studied (1×10⁶ cells), intraperitoneal MSCs effectively restored inspiratory capacity, compliance of the respiratory system (Crs), static compliance (Cst), elastance of the respiratory system (Ers), and tissue elastance (H) to normoxic levels (Table 4). Intraperitoneal MSCs further significantly reduced the total lung capacity (A) and area of the PV loop of hyperoxic animals, reaching levels below those seen in normoxic animals. Even at the highest dose, intraperitoneal MSCs had no obvious effects on airway resistance (Rn), resistance of the respiratory system (Rrs), or eta (Tables 3 and 4). Selected functional parameters are shown in FIG. 2.

Pressure-volume loops were generated using the data provided by the stepwise PVr-P maneuver. For the sake of clarity, only data for PBS-treated normoxic and hyperoxic controls and for hyperoxic animals treated with high-dose MSCs are shown in FIG. 3. In PBS-treated control animals, neonatal hyperoxia exposure was associated with an upward shift of the PV curve in adulthood, consistent with an emphysematous pulmonary phenotype (FIG. 3). Intranasal MSC administration had no obvious effects on position or shape of the PV loop at any dose; in fact, the PV loop of hyperoxia-exposed, IN MSC-treated animals at low, medium or high doses showed almost perfect alignment with that of hyperoxic controls (FIG. 3, left, showing high-dose IN MSC). In contrast to the lack of effects seen following IN MSC administration, intraperitoneal high-dose MSC administration was associated with a dramatic downward shift of the hyperoxic PV loops to reach normoxic levels (FIG. 3, right). This downward shift was associated with closer approximation of inspiration and expiration curves, consistent with the reduction in area of the PV loop described above. The PV loops of animals treated with low- or medium-dose IP MSCs were positioned intermediate between those of PBS-treated hyperoxic controls and those of animals treated with high-dose IP MSCs (not shown).

Taken together, these lung mechanics studies suggest that neonatal hyperoxia exposure leads to an emphysema-like functional phenotype in adulthood, characterized by increased compliance and decreased elasticity (diminished tissue recoil). Intraperitoneal administration of high-dose (1×10⁶/pup) MSCs during the neonatal period resulted in restoration or preservation of normal lung compliance and elasticity 8 weeks post-transplantation, suggestive of normalization of tissue recoil. Irrespective of dose, intranasal MSCs had no obvious effects on lung function.

Analysis of Early Pulmonary and Systemic Distribution of MSCs in Newborn Mice Following Intranasal or Intraperitoneal Administration

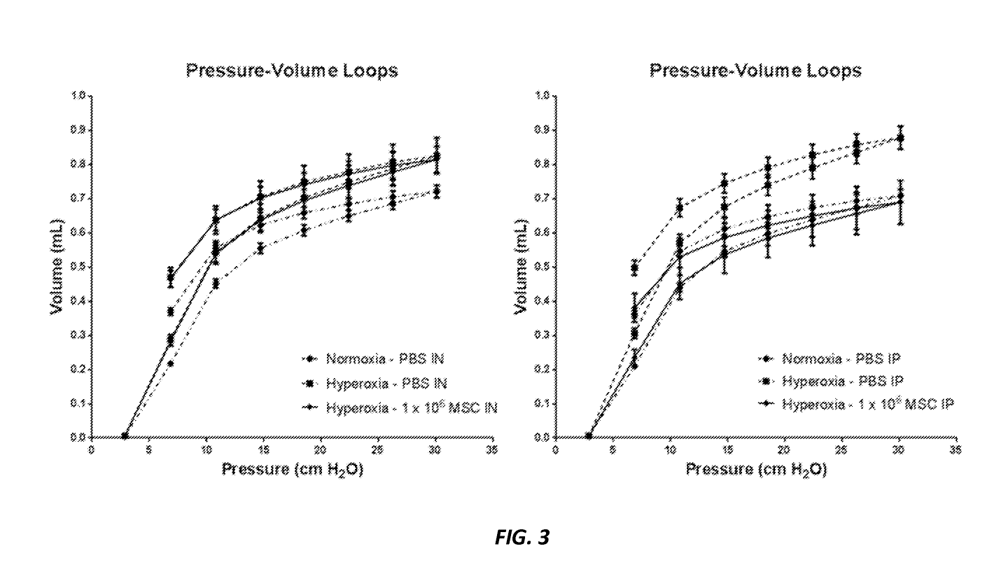

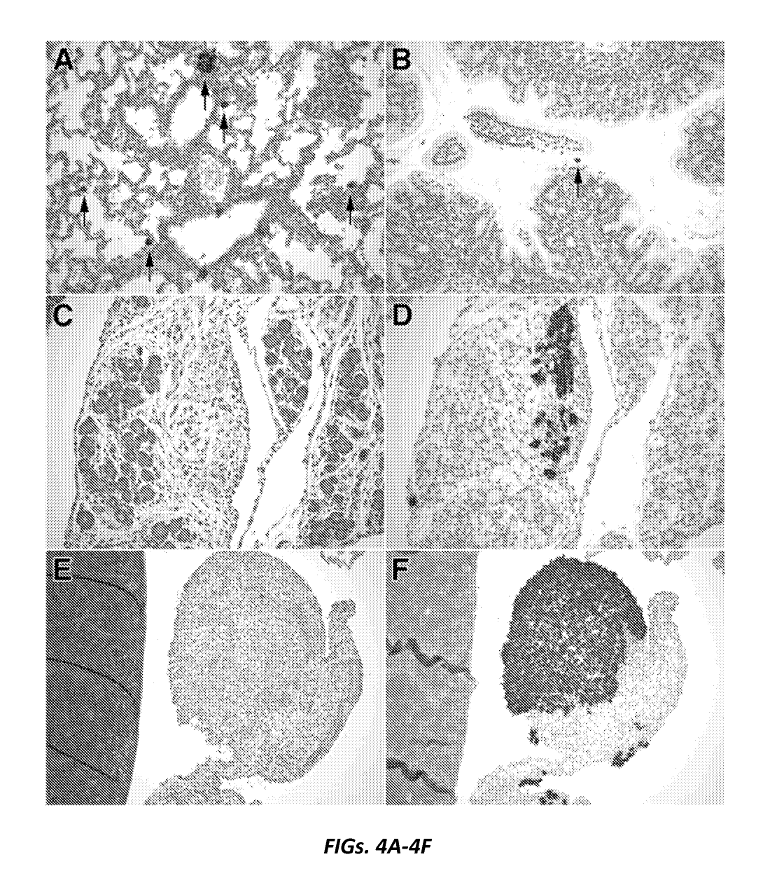

Cell fate and distribution were studied within 48 h after IN and IP administration in a small number of animals (N=4 per delivery route). The inventors sought to understand the mechanisms underlying the observed functional effects of MSCs following intraperitoneal delivery and the lack of effect following intranasal delivery. The dispersion of IN or IP administered MSCs (1×10⁶) to lungs and selected organs was monitored by anti-human vimentin immunohistochemistry. Intranasal administration of MSCs in newborn mice resulted in even and effective cellular distribution in both lungs (FIG. 4A), confirming previous results with murine whole bone marrow or human CD34+ hematopoietic progenitor cells. There was no obvious histopathologic evidence of an associated inflammatory response. No human vimentin-positive cells were detected in liver, spleen, bone marrow or kidneys (not shown). As expected following IN inoculation, very rare human vimentin-positive cells were detected in the lumen of the gastrointestinal tract, reflective of occasional spillage of cells from the upper respiratory tract (FIG. 4B).

The short-term pulmonary and systemic distribution of MSCs was studied 48 hours following intraperitoneal administration. No vimentin-immunoreactive MSCs were detected within the lung parenchyma of any of 4 animals examined. Similarly, no human vimentin immunoreactive cells were seen in spleen, liver, kidneys or bone marrow (not shown). However, examination of the remaining abdominal contents, subjected to histologic analysis in toto, revealed the presence of MSCs in all animals, stably embedded in peritoneal or retroperitoneal organs and soft tissues. These human vimentin-positive cells were detected as single cells, small clusters or even distinct, highly cellular nodular aggregates displaying brisk proliferative activity (FIGS. 4C-4F). Omission of primary anti-vimentin antibody abolished all immunoreactivity. Anti-human vimentin staining of tissues of control newborn mice that did not receive MSCs was uniformly negative (not shown).

Analysis of Long-Term Engraftment of MSCs or their Progeny in Lungs and Other Organs of Newborn Mice

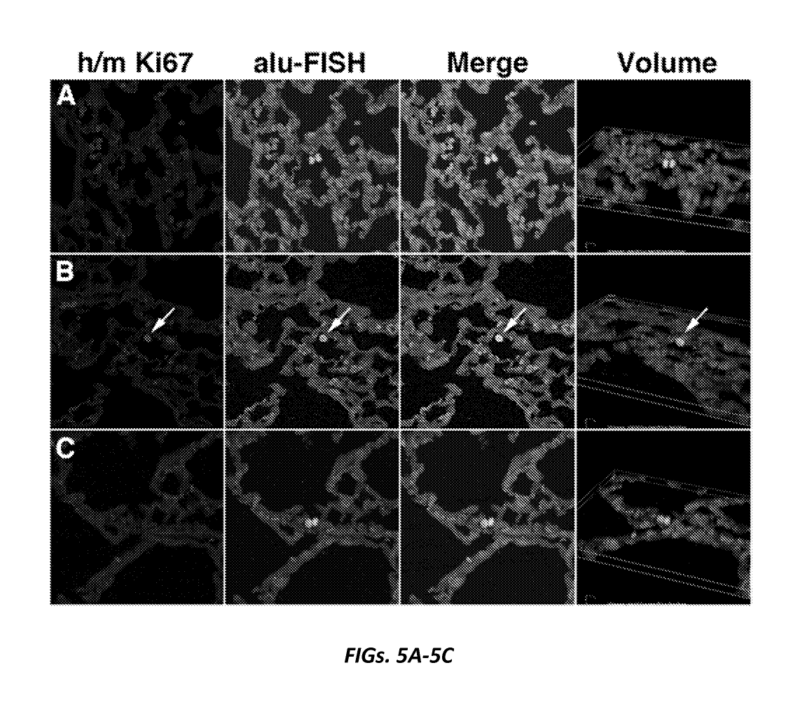

The pulmonary and systemic presence of MSCs or MSC-derived cells at 8 weeks post-transplantation was studied by human-specific alu-FISH analysis. These studies were limited to animals treated with the high-dose (1×10⁶ cells/pup) regimen. Rare alu-FISH-positive nuclei were identified in the lungs of all MSC-recipient animals, regardless of delivery route (FIG. 5). The engrafted cells appeared to be randomly distributed in central and peripheral lung parenchyma without obvious topographic predilection. Most MSC-derived cells were single, although occasionally alu-FISH-positive cells were seen as doublets or triplets, suggestive of recent clonogenic expansion (FIGS. 5A and 5C). In support of this interpretation, occasional proliferative activity was detected in engrafted MSC-derived cells by combined alu-FISH analysis and Ki67 immunostaining, in intranasal as well as intraperitoneal treatment groups (FIG. 5B). In view of the low numerical density of MSC-derived cells in either delivery group, no formal quantitation was performed. The systemic presence of MSC-derived cells was studied by vimentin staining and alu-FISH analysis of selected organs. No human-derived cells were detected in random sections of liver, spleen, heart, or kidneys (not shown).

Taken together, the short- and long-term cellular distribution studies indicate that intranasal administration results in homogenous distribution solely within the lung parenchyma. Following intraperitoneal administration, cells tended to remain in the peritoneum or retroperitoneum, although eventually some disseminated to and were retained in the lungs. The viability of cells in the peritoneal cavity and their virtual absence in lungs immediately after transplantation suggest that any pulmonary effects from intraperitoneal MSC administration are likely attributable to systemic paracrine effects, rather than to direct structural integration.

Analysis of Cellular Composition of Bronchoalveolar Lavage Fluid 8 Weeks Post-Transplantation

The paucity of intrapulmonary human-derived cells detected 8 weeks post-transplantation in this study supports the growing notion that the pulmonary effects of stem cells, regardless of cell type, are based on paracrine, anti-inflammatory and/or immunomodulatory, activities of the cells. To begin to explore this potential role in the transplant model, the morphology of cells in the bronchoalveolar lavage fluid was examined 8 weeks post-transplantation.

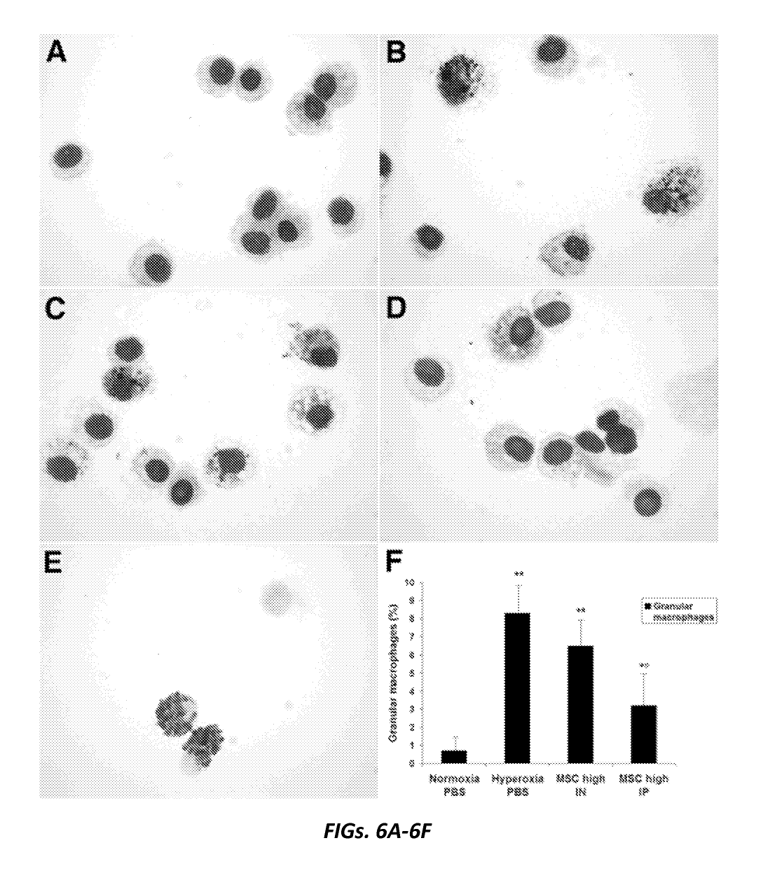

The cellular composition of the lavage fluid was equivalent between the various treatment groups: in all groups, alveolar macrophages accounted for >95% of cells. Variable numbers of scattered lymphocytes and rare eosinophils comprised the remaining 5%. While the cellular composition was similar, the appearance of alveolar macrophages varied between the various treatment groups. Compared with normoxic controls, the lavage fluid of hyperoxia-exposed control animals contained a relatively high proportion of macrophages containing cytoplasmic granules of heterogeneous size and shape (FIGS. 6A-6B). These cytoplasmic granules were morphologically consistent with hemosiderin; positive Perls stain confirmed the iron content of the cytoplasmic granules (FIG. 6E). The presence of hemosiderin-laden macrophages in MSC-treated animals (high-dose) was compared (FIGS. 6C-6D) and it was observed that the fraction of hemosiderin-laden macrophages was significantly lower in animals treated with IP MSCs than in hyperoxic controls (FIG. 6F). The lower fraction of hemosiderin-laden macrophages 8 weeks post-transplantation suggests that acute, hemorrhagic lung injury induced by hyperoxia exposure may have been shortened or attenuated by high-dose IP MSC treatment.

### DISCUSSION

In this example, the inventor performed a systematic comparative analysis of the functional and morphologic effects of cultured human cord tissue MSCs, administered via either the systemic (intraperitoneal, IP) or intrapulmonary (intranasal, IN) route. Cells (0.1, 0.5 or 1×10⁶ cells/pup) were administered during the newborn period (P5) to immune-suppressed SCID-beige mice with hyperoxia-induced neonatal lung injury; the functional and morphologic/morphometric outcomes were assessed 8 weeks post-transplantation.

Lung mechanics were assessed by the invasive forced oscillation technique (FlexiVent), which provides accurate and reproducible estimation of critical parameters such as compliance, elastance, and resistance of the rodent respiratory system (Vanoirbeek J A, et al.: Noninvasive and invasive pulmonary function in mouse models of obstructive and restrictive respiratory diseases, Am J Respir Cell Mol Biol 2010, 42:96-104). In agreement with similar studies by Yee et al. (Neonatal oxygen adversely affects lung function in adult mice without altering surfactant composition or activity, Am J Physiol Lung Cell Mol Physiol 2009, 297:L641-649), it was first established that one-week of hyperoxia exposure at 90% O₂ in the newborn period has long lasting functional effects in adulthood, characterized by significantly increased lung compliance and diminished elastance, associated with an upward shift of the pressure-volume loops. Increased lung compliance/reduced elastance is a functional hallmark of the loss of elastic recoil seen in pulmonary emphysema (Vanoirbeek J A, et al.: Noninvasive and invasive pulmonary function in mouse models of obstructive and restrictive respiratory diseases, Am J Respir Cell Mol Biol 2010, 42:96-104; Shim Y M, et al.: Role of LTB(4) in the pathogenesis of elastase-induced murine pulmonary emphysema, Am J Physiol Lung Cell Mol Physiol 2010, 299:L749-759), and entirely consistent with the emphysema-like phenotype with simplified and enlarged airspaces typical of neonatal hyperoxia exposure in rodents (Mao Q, et al.: The Fas system confers protection against alveolar disruption in hyperoxia-exposed newborn mice, Am J Respir Cell Mol Biol 2008, 39:717-729; Fritzell J A, Jr., et al.: Fate and effects of adult bone marrow cells in lungs of normoxic and hyperoxic newborn mice, Am J Respir Cell Mol Biol 2009, 40:575-587; Crapo J D, et al.: Structural and biochemical changes in rat lungs occurring during exposures to lethal and adaptive doses of oxygen, Am Rev Respir Dis 1980, 122:123-143), and bronchopulmonary dysplasia in human preterm infants (Husain A N, et al.: Pathology of arrested acinar development in postsurfactant bronchopulmonary dysplasia, Hum Pathol 1998, 29:710-717; Jobe A J: The new BPD: an arrest of lung development, Pediatr Res 1999, 46:641-643; De Paepe M E, et al.: Growth of pulmonary microvasculature in ventilated preterm infants, Am J Respir Crit Care Med 2006, 173:204-211).

Intraperitoneal administration of human cultured MSCs to hyperoxia-exposed newborn mice resulted in a dose-dependent decrease in lung compliance (and corresponding increase in elastance) by 8 weeks post-transplantation. At the highest dose studied (1×10⁶ MSCs/animal), IP MSC administration effectively restored/normalized lung compliance, elastance and pressure-volume loops to normoxic control levels. The exact biochemical/structural correlates of the observed increased lung compliance/elastic recoil associated with IP MSC administration remain to be determined. Pulmonary elastic recoil is approximately equally determined by two main anatomic attributes of the lung parenchyma: the elastic properties of its interstitium and the unique structure and complexity of the liquid-filled alveolar network (Shiner R, Steier J: Lung function tests. Churchill Livingstone Elsevier, New York 2013). As determined in this study, high-dose IP MSC delivery caused only a mild increase in alveolar septation (decrease in mean cord length). More strikingly, however, high-dose intraperitoneal MSC delivery was associated with a significant increase in mean septal wall thickness, suggesting that the normalizing functional effects of IP MSCs were mediated, in large part, by modification of abundance and/or composition of the interstitial extracellular matrix, leading to improved pulmonary elastic recoil.

The apparent matrix-modulating effects of MSCs following IP administration are consistent with the functions of these mesenchymally active, potentially profibrotic cells (52. Pierro M, Thebaud B: Mesenchymal stem cells in chronic lung disease: culprit or savior?, Am J Physiol Lung Cell Mol Physiol 2010, 298:L732-734). Mesenchymal stromal cells have been shown to stimulate lung fibroblast proliferation and matrix production, two characteristics of fibroproliferative lung disease (Salazar K D, et al.: Mesenchymal stem cells produce Wnt isoforms and TGF-beta1 that mediate proliferation and procollagen expression by lung fibroblasts, Am J Physiol Lung Cell Mol Physiol 2009, 297:L1002-1011). While the relatively short-term (8-week) effects of intraperitoneal MSCs in this study appeared to be beneficial and restored tissue recoil to baseline levels, the longer-term matrix-modulating effects of these mesenchymally active cells deserve close monitoring. Available preclinical data from various lines of investigation suggest that MSC administration may contribute to pulmonary fibrosis, at least in part by differentiation into myofibroblasts (Epperly M W, et al.: Bone marrow origin of myofibroblasts in irradiation pulmonary fibrosis, Am J Respir Cell Mol Biol 2003, 29:213-224; Sun Z, et al.: Activated Wnt signaling induces myofibroblast differentiation of mesenchymal stem cells, contributing to pulmonary fibrosis, Int J Mol Med 2014, 33:1097-1109; Tang N, et al.: Lysophosphatidic acid accelerates lung fibrosis by inducing differentiation of mesenchymal stem cells into myofibroblasts, J Cell Mol Med 2014, 18:156-169).

Whereas IP MSC delivery was found to have significant effects, IN delivery of MSCs from the same batch, to the same host litter, and at similar doses did not have any noticeable effects on lung mechanics. Specifically, IN inoculation of MSCs at doses ranging between 0.1 and 1×10⁶ cells/pup did not affect lung compliance, elastance, pressure-volume loops, or resistance. Similarly, IN inoculation had no effects on alveolar remodeling or septal wall thickness. These results are in disagreement with other studies that reported beneficial effects of intratracheal MSCs on alveolar septation, lung vascular injury, and/or exercise intolerance in immunocompetent hyperoxia-exposed newborn rats (van Haaften T, et al.: Airway delivery of mesenchymal stem cells prevents arrested alveolar growth in neonatal lung injury in rats, Am J Respir Crit Care Med 2009, 180:1131-1142; Chang Y S, et al.: Human umbilical cord blood-derived mesenchymal stem cells attenuate hyperoxia-induced lung injury in neonatal rats, Cell Transplant 2009, 18:869-886; Pierro M, et al.: Short-term, long-term and paracrine effect of human umbilical cord-derived stem cells in lung injury prevention and repair in experimental bronchopulmonary dysplasia, Thorax 2013, 68:475-484). The reasons for these apparent discrepancies remain unclear. Differences in the timing of cell administration, model of neonatal lung injury, recipient strain, MSC cell processing, culturing, and donor effects may be implicated.

Evidence is continuously accumulating suggesting multiple immunomodulatory and anti-inflammatory paracrine effects of MSCs, either mediated directly by peptides/growth factors, or by transfer of exosomes, microvesicles, or organelles [reviewed in Weiss D J: Stem cells, cell therapies, and bioengineering in lung biology and diseases. Comprehensive review of the recent literature 2010-2012, Ann Am Thor Soc 2013, 10:545-97; and Weiss D J: Concise review: current status of stem cells and regenerative medicine in lung biology and diseases, Stem Cells 2014, 32:16-25]. Several of the findings herein support the notion that the observed effects of IP MSCs may be attributable to indirect, paracrine, anti-inflammatory effects. In agreement with observations by others (Kassmer S H, Krause D S: Detection of bone marrow-derived lung epithelial cells, Exp Hematol 2010, 38:564-573), structural integration of MSCs or their progeny into the lung parenchyma was only sporadic. Instead, the stable engraftment and brisk proliferative activity observed in peritoneal and retroperitoneal MSC implants studied immediately post-transplantation suggests these cells may have been capable of secretory activity for a prolonged time period following administration.

Parenthetically, the cellular composition of the bronchoalveolar lavage fluid was equivalent between normoxic or hyperoxia-exposed controls and MSC-treated hyperoxia-exposed animals, consisting almost exclusively of alveolar macrophages in all groups. Closer examination revealed interesting differences between these groups with respect to the cellular features. Hemosiderin is a product of hemoglobin degradation, thus hemosiderin-laden alveolar macrophages are generally considered to be reflective of past intraalveolar hemorrhage, such as may be seen in association with acute lung injury. As expected, the fraction of hemosiderin-containing macrophages was much higher in hyperoxia-exposed animals than in normoxic controls. Interestingly, the fraction of hemosiderin-laden macrophages was significantly lower in IP MSC-treated animals than in hyperoxic controls, suggesting MSC administration in the newborn period may have attenuated or shortened the acute lung injury phase.

In summary, the results shown herein suggest that intraperitoneal (systemic) administration of cultured human MSCs at high dose has the capacity to restore the lung mechanics (compliance, recoil) of hyperoxia-exposed newborn mice to normal levels, presumably by modification of the interstitial matrix. The brisk initial peritoneal engraftment of MSCs and low pulmonary engraftment levels suggest that these effects were mediated by paracrine factors, rather than by direct structural regeneration of the injured lung parenchyma by MSCs or their progeny. In contrast to the striking beneficial effects achieved by intraperitoneal administration, intranasal inoculation of MSCs at the same dose had no effects on lung function or morphology. This study provides evidence of the beneficial therapeutic potential of MSCs in neonatal lung diseases as well as in adult lung diseases characterized by diminished tissue recoil, such as COPD/emphysema.

What is claimed is:

1. A method for treating or preventing a lung disorder in a subject in need thereof, comprising administering a therapeutically effective amount of a population of isolated or enriched umbilical cord tissue-derived mesenchymal stem cells to said subject via a systemic route.

2. The method of claim 1, wherein the systemic route is intraperitoneal administration.

3. The method of claim 1, wherein the systemic route is intravenous injection.

4. The method of claim 1, wherein the lung disorder is chronic lung disease of the newborn.

5. The method of claim 1, wherein the subject is an infant or a preterm infant.

6. The method of claim 1, further comprising selecting a subject who is suffering from a lung disorder prior to administering the population of isolated or enriched umbilical cord tissue-derived mesenchymal stem cells to the subject.

7. The method of claim 1, wherein the population of isolated or enriched umbilical cord tissue-derived mesenchymal stem cells are expanded or cultured ex vivo prior to administration to the subject.

8. The method of claim 1, wherein the mesenchymal stem cells are selected based on positive expression of one or more of CD73, CD90, and CD105.

9. The method of claim 8, wherein the mesenchymal stem cells are selected based on negative expression of one or more of CD34, CD45, CD14, CD19, and HLA-DR.

10. The method of claim 1, wherein the population of isolated or enriched umbilical cord tissue-derived mesenchymal stem cells are autologous cells.

11. The method of claim 1, wherein the population of isolated or enriched umbilical cord tissue-derived mesenchymal stem cells are allogeneic cells obtained from one or more donors.

12. The method of claim 1, further comprising administering at least one therapeutic agent.

13. The method of claim 12, wherein the at least one therapeutic agent enhances homing, engraftment, or survival of the population of isolated or enriched umbilical cord tissue-derived mesenchymal stem cells.
